(19) United States
(12) Patent Application Publication
(10) Pub. No.: US 2021/0283144 A1
(43) Pub. Date: Sep. 16, 2021

(54) MODULATION OF SEX STEROID HORMONE PRODUCTION AND ACTIVITY FOR TREATING AND PREVENTING HERNIAS

(71) Applicant: Northwestern University, Evanston, IL (US)

(72) Inventors: Serdar E. Bulun, Chicago, IL (US); Hong Zhao, Wilmette, IL (US)

(21) Appl. No.: 17/198,558

(22) Filed: Mar. 11, 2021

Related U.S. Application Data
(60) Provisional application No. 62/988,218, filed on Mar. 11, 2020.

(51) Int. Cl. A61K 31/5685; A61K 31/4535; A61K 31/566; A61K 31/4196; A61K 9/00; A61P 43/00; A61K 31/56; A61F 2/00

(52) U.S. Cl. CPC A61K 31/5685; A61K 31/4535; A61K 31/566; A61K 31/4196; A61K 9/0014; A61F 2250/0067; A61K 9/0019; A61P 43/00; A61K 31/56; A61F 2/0063; A61K 9/0053

(57) ABSTRACT

Disclosed are methods and compositions for treating and/or preventing hernias in subjects in need thereof. The disclosed methods of treatment and/or preventing may include methods for treating and/or preventing hernias in subject in need thereof by administering to the subject a therapeutic agent that modulates sex steroid hormone production and/or activity in the subject. Hernias treated and/or prevented by the disclosed methods may include, but are not limited to, inguinal hernias, femoral hernias, umbilical hernias, hiatal hernias, incisional hernias, and diastasis recti.

### CROSS-REFERENCE TO RELATED PATENT APPLICATIONS

This application claims the benefit of priority under 35 U.S.C. § 119(e) to U.S. Provisional Application No. 62/988,218, filed on Mar. 11, 2020, the content of which is incorporated herein by reference in its entirety.

### STATEMENT REGARDING FEDERALLY SPONSORED RESEARCH OR DEVELOPMENT

This invention was made with government support under grant HD038691 and DK121529 awarded by the National Institutes of Health. The government has certain rights in the invention.

### BACKGROUND

The field of the invention relates to methods and compositions for treating and/or preventing hernias in subjects in need thereof. In particular, the field of the invention relates to methods for treating and/or preventing hernias in subject in need thereof by administering to the subject a therapeutic agent that modulates sex steroid hormone production and/or activity in the subject.

Inguinal repair is the most common general surgical procedure performed in the US today (600,000/year). More than 1 in 4 men will undergo inguinal hernia repair during their lifetime. Annual health care costs directly attributable to inguinal hernia exceed $2.5 billion in the US. The only effective treatment is surgical repair. Although surgery can cure the majority of inguinal hernias in otherwise healthy patients, recurrent hernias in very elderly men or men with severe diabetes and liver failure challenge surgeons due to high morbidity and mortality and high rate of complications (e.g., wound infection, recurrence, and long-term severe pain). The high prevalence of inguinal hernias, combined with poor surgical outcomes in recurrent hernias and high associated costs, reveals an unmet need for developing preventive and therapeutic strategies in high-risk populations. We found the conversion of testosterone to estradiol (E2) by the estrogen biosynthesis enzyme in lower abdominal muscle tissue (LAMT) causes intense fibrosis, leading to muscle atrophy and inguinal hernia. Modulation of estrogen action entirely prevents this phenotype and reverses mild to severe hernias. Our findings indicate that sex steroid hormone production and/or activity can be modulated in order to treat inguinal hernias and other hernias including femoral, umbilical, hiatal, incisional hernias, and diastasis recti.

### SUMMARY

Disclosed are methods and compositions for treating and/or preventing hernias in subjects in need thereof. The disclosed methods of treatment and/or preventing may include methods for treating and/or preventing hernias in subject in need thereof by administering to the subject a therapeutic agent that modulates sex steroid hormone production and/or activity in the subject. Hernias treated and/or prevented by the disclosed methods may include, but are not limited to, inguinal hernias, femoral hernias, umbilical hernias, hiatal hernias, incisional hernias, and diastasis recti.

In the disclosed methods, a subject in need thereof may be administered a therapeutic agents that treats and/or prevents a hernia in the subject. The therapeutic agents that are administered to a subject in the disclosed methods typically modulate the production and/or activity of sex steroid hormones in the subject. The therapeutic agents may modulate the production and/or activity of sex steroid hormones that include, but are not limited to, estrogen and androgens. The therapeutic agents may modulate the production, degradation, and/or activity of cell receptors for sex steroid hormones that include, but are not limited to, cell receptors for estrogen and cell receptors for androgens. Suitable therapeutic agents for use in the disclosed methods may include anti-estrogen therapeutics, aromatase inhibitors, androgens, and androgen stimulators.

The disclosed therapeutic agents that modulate production may be formulated for administration to a subject having or at risk for developing a hernia. The disclosed therapeutics may be formulated for systemic and/or local administration at the site of a hernia or a site at risk for developing a hernia.

### BRIEF DESCRIPTION OF THE FIGURES

FIG. 1. Overall hypothesis. Aims are labeled in the model. Roles of aromatase, estrogen, and estrogen receptor α (ERα) in fibroblast proliferation, fibrosis, and hernia formation.

FIG. 2. Schematic of mouse abdominal muscle anatomy. Solid trapezoid shaped region indicates normal LAMT in wild-type (WT) mice. Dashed line indicates fibrotic LAMT that comprises the hernia wall contiguous with the scrotum of humanized aromatase (Arom^{hum}) mice.

FIG. 3. Human aromatase promoter usage and expression in Arom^{hum} mice. (A) Human BAC construct used to generate an Arom^{hum} mouse line, which contains a nearly full-length innate 5′-regulatory region and full-length coding and 3′-regions of the human aromatase gene. (B) Exon-specific RT-PCR confirmed different promoters drive human aromatase expression in the abdominal muscle of Arom^{hum} mice (Control: GAPDH mRNA). (C) Human aromatase mRNA levels in upper abdominal muscle tissue (UAMT) and LAMT of WT and Arom^{hum} mice. n=8 mice/group. *p<0.05.

FIG. 4. (A) Serum estradiol (E2) and (B) LAMT E2 were measured by liquid chromatography-tandem mass spectrometry (LC-MS²) assay after letrozole (let) treatment (n=10/group). *p<0.05. **p<0.01. Veh, vehicle.

FIG. 5. (A) LAMT defects and inguinal hernia (arrows). (B) Hernia incidence in the Arom^{hum} mice. n=29. (C) The aromatase inhibitor letrozole prevents hernia formation in Arom^{hum} mice. Veh, vehicle; Let, letrozole. n=10.

FIG. 6. (A) H&E staining showing disordered proliferation of fibroblasts and excessive extracellular matrix (ECM) deposition (fibrosis) and atrophy of myocytes (arrow) in LAMT of 24-week-old Arom^{hum} mice. (B) Masson's trichrome staining shows that aromatase inhibitor letrozole prevented LAMT atrophy and fibrosis. Veh, vehicle; Let, letrozole. n=10.

FIG. 7. Increased fibroblast proliferation and collagen formation are stimulated by E2 and inhibited by letrozole in LAMT of Arom^{hum} mice. (A) Ki67 immunostaining comparing proliferation in LAMT fibroblasts of WT and Arom^{hum} mice. (B) Image analysis of Ki67-positive stromal nuclei in UAMT and LAMT of WT and Arom^{hum} mice (n=10). (C) Cell proliferation marker PCNA and ECM marker Col1a1 protein levels in primary LAMT fibroblasts of WT mice after E2 treatment. Data are representative of 3 independent experiments. (D) Total collagen content in UAMT and LAMT of WT and Arom^{hum} mice measured by hydroxyproline assay (n=5). (E) Percent of Ki67-positive stromal cells in LAMT of WT or Arom^{hum} mice treated with vehicle (Veh) or letrozole (Let). n=10. *p<0.05.

FIG. 8. mRNA levels of (A) estrogen-responsive genes (Ccnd1 and Greb1) in primary fibroblasts and (B) pro-fibrotic genes (Kiss1, Ren1, Emb, Timp1, Spon2, and Eln) in tissue lysates of UAMT and LAMT from WT and Arom^{hum} mice. n=10. *p<0.05. **p<0.01.

FIG. 9. Nuclear ERα expression in UAMT and LAMT of Arom^{hum} mice. (A) Nuclear ERα in UAMT and LAMT of WT and Arom^{hum} mice assessed by IHC staining. (B) A total of 2000 nuclei were counted in sections to calculate the percent of ERα-positive stromal nuclei in UAMT and LAMT of WT and Arom^{hum} mice (n=8-10). (C) ERα protein levels in primary fibroblasts of UAMT and LAMT in WT mice. Data are representative of 3 independent experiments. **p<0.01. *p<0.05.

FIG. 10. mRNA levels of estrogen-responsive genes [Pgr (A, E) and Greb1 (B, F)] and fibrotic genes [Spon2 (C, G), Kiss1 (D), and Eln (H)] in LAMT primary fibroblasts fromArom^{hum} mice after treatment with 100 nM ICI 182,780 (ICI) or 10 μM MPP in the presence or absence of E2 (10 nM). Cells were pretreated with ICI 182,780 or MPP for 2 hours before the addition of E2. Veh, vehicle. *P<0.05, **P<0.01. GAPDH mRNA levels served as controls.

FIG. 11. (A) Working model for Hypothesis 1b. CIS: cis-regulatory elements. FAFs: fibrosis-associated fibroblasts. (B) Primary LAMT fibroblasts from Arom^{hum} mice were formaldehyde-crosslinked and chromatin was prepared and digested. ChIP DNA was purified and then separated on a 1% agarose gel. The majority of chromatin was digested to 1 to 4 nucleosomes in length (150 to 600 bp), which is favorable for ChIP-seq analyses.

FIG. 12. ERα is expressed in Fsp1-positive fibroblasts of LAMT. LAMTs were analyzed by immunofluorescence staining for Fsp1 and ERα.

FIG. 13. mRNA levels of estrogen-responsive genes [Pgr (A) and Greb1 (B)] and fibrotic genes [Kiss1 (C) and Spon2 (D)] in LAMT primary fibroblasts from Arom^{hum} mice after siRNA-mediated knockdown of ERα in the presence or absence of E2 (10 nM). Veh, vehicle. *P<0.05, **P<0.01.

FIG. 14. (A) Masson's trichrome staining (MTS) and immunoreactive (B) ERα and (C) Ki67 in LAMT of hernia-free men and hernia patients. Yellow arrows indicate atrophic myocytes. Red arrows indicate positive (brown) staining. n=6 per group. *p<0.05. (D) ERα H-score for hernia-free men and hernia patients. (E) % of Ki67-positive nuclei in fibroblasts of hernia-free men and hernia patients.

FIG. 15. ER-dependent E2 antagonist fulvestrant rescues lower abdominal muscle tissue (LAMT) fibrosis and myocyte atrophy and prevents the development of scrotal hernias in Arom^{hum} mice. 4-week-old mice were treated with placebo or fulvestrant for 12 weeks. (A) Schematic diagram depicts the schedule of drug administration. (B) The area of scrotal hernia in Arom^{hum} mice. (C) Representative photomicrographs of Masson's trichrome-stained LAMT sections of WT and Arom^{hum} mice. Quantification of the percentage of connective tissue or fibrotic area (D) or myofiber area (E) in WT and Arom^{hum} mice. 10 representative high-power fields were analyzed in each tissue. One-way ANOVA with Tukey's multiple comparison test, *p<0.05, ***p<0.001. Scale bars, 50 μm. n=5 mice per group.

FIG. 16. Aromatase inhibitor letrozole reverses the small to moderate size of scrotal hernias and rescues LAMT fibrosis and myocyte atrophy in Arom^{hum} mice. Arom^{hum} mice with small to moderate size of hernias were treated with placebo or letrozole for 12 weeks. (A) Schematic diagram depicts the schedule of drug administration. (B) The area of scrotal hernia in Arom^{hum} mice. (C) Representative photomicrographs of Masson's trichrome-stained LAMT sections of WT and Arom^{hum} mice. Quantification of the percentage of connective tissue or fibrotic area (D) or myofiber area (E) in WT and Arom^{hum} mice. 10 representative high-power fields were analyzed in each tissue. One-way ANOVA with Tukey's multiple comparison test, **p<0.01, ***p<0.001. Scale bars, 50 pin. n=5 mice per group.

FIG. 17. E2/ER antagonist raloxifene reverses the small to moderate size of scrotal hernias in Arom^{hum} mice. Arom^{hum} mice with small to moderate size of hernias were treated with placebo or letrozole for 3 weeks. (A) Schematic diagram depicts the schedule of drug administration. (B) The area of scrotal hernia in Arom^{hum} mice.

FIG. 18. E2/ER antagonist fulvestrant reverses the large size of scrotal hernias and rescues LAMT fibrosis and myocyte atrophy in Arom^{hum} mice. Arom^{hum} mice with large size of hernias were treated with placebo or fulvestrant for 12 weeks. (A) Schematic diagram depicts the schedule of drug administration. (B) The area of scrotal hernia in Arom^{hum} mice. (C) Representative photomicrographs of Masson's trichrome-stained LAMT sections of WT and Arom^{hum} mice. Quantification of the percentage of connective tissue or fibrotic area (D) in WT and Arom^{hum} mice. 10 representative high-power fields were analyzed in each tissue. (E) Hydroxyproline content in LAMT of WT and Arom^{hum} mice treated with fulvestrant. One-way ANOVA with Tukey's multiple comparison test, ***p<0.001, ****p<0.0001. n=5 mice per group.

FIG. 19. Aromatase inhibitor letrozole reverses the large size of scrotal hernias in Arom^{hum} mice. Arom^{hum} mice with large size of hernias were treated with placebo or letrozole for 12 weeks. (A) Schematic diagram depicts the schedule of drug administration. (B) The area of scrotal hernia in Arom^{hum} mice.

FIG. 20. E2/ER antagonist raloxifene reverses the large size of scrotal hernias in Arom^{hum} mice. Arom^{hum} mice with large size of hernias were treated with placebo or raloxifene for 3 weeks. (A) Schematic diagram depicts the schedule of drug administration. (B) The area of scrotal hernia in Arom^{hum} mice.

FIG. 21. Dihydrotestosterone (DHT) decreases the volume of scrotal hernias in Arom^{hum} mice. 3-week-old mice were treated with vehicle (Veh) or DHT for 53 days. (A) Schematic diagram depicts the schedule of drug administration. (B) The volume of scrotal hernia in Arom^{hum} mice. *p<0.005, n=5 mice per group.

### DETAILED DESCRIPTION

The present invention is described herein using several definitions, as set forth below and throughout the application.

### Definitions

Unless otherwise specified or indicated by context, the terms “a”, “an”, and “the” mean “one or more.” For example, “an inhibitor of tumor cell aggregation” should be interpreted to mean “one or more inhibitors of tumor cell aggregation.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, a “subject” may be interchangeable with “patient” or “individual” and means an animal, which may be a human or non-human animal, in need of treatment, for example, treatment by include administering a therapeutic amount of one or more therapeutic agents that modulate the production and/or activity of a sex steroid hormone in a subject.

A “subject in need of treatment” may include a subject having or at risk for developing a hernia. In particular, a subject in need of treatment may include a subject having or at risk for developing a hernia selected from one or more of an inguinal hernia, a femoral hernia, an umbilical hernia, a hiatal hernia, diastasis recti, and/or an incisional hernia.

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art. In the disclosed methods, a subject in need thereof may be administered an effective amount of a therapeutic agent for treating and/or preventing a hernia in the subject.

The disclosed therapeutic agents may be effective for modulating the production and/or activity of a sex steroid hormone in a subject. As disclosed herein, the term “modulation” may include increasing production (and/or concentration) and/or activity of the sex steroid hormone in a subject, or conversely, decreasing production and/or activity of the sex steroid hormone in a subject. For example, modulation of production of sex steroid hormone may include administering a therapeutic agent that inhibits production of a sex steroid hormone in the subject such as an aromatase inhibitor that inhibits the production of estrogen in the subject. Modulation of activity of a sex steroid hormone in a subject may include administering a therapeutic agent that modulates or degrades a receptor for estrogen in the subject. Modulation of production and/or activity of a sex steroid hormone in a subject may include administering an androgen and/or an androgenic agent to the subject.

The therapeutic agents utilized in the methods disclosed herein may be formulated as pharmaceutical compositions that include: (a) a therapeutically effective amount of one or more of the therapeutic agents as disclosed herein; and (b) one or more pharmaceutically acceptable carriers, excipients, or diluents.

Modulation of Sex Steroid Hormone Production and Activity for Treating and Preventing Hernias

The disclosed subject matter relates to methods and compositions for treating and/or preventing hernias in subjects in need thereof. Suitable subjects for the disclosed methods may include subjects having or at risk for developing a hernia. Hernias treated and/or prevented by the disclosed methods may include, but are not limited to, an inguinal hernia, a femoral hernia, an umbilical hernia, a hiatal hernia, an incisional hernia, and a diastasis recti.

In the disclosed methods, a subject in need thereof is administered one or more therapeutic agents that modulate (e.g., increase and/or decrease) the production (and/or concentration) and/or activity of one or more sex steroid hormones in the subject. In particular, the therapeutic agents administered in the disclosed methods may modulate the production and/or activity of sex steroid hormones that include, but are not limited to estrogens and androgens.

In some embodiments of the disclosed methods, a subject having or at risk for developing a hernia is administered a therapeutic agent that inhibits the production and/or activity of estrogen in the subject. In particular embodiments, the therapeutic agent is an anti-estrogen therapeutic agent that inhibits the activity of estrogen. In some embodiments, the anti-estrogen therapeutic agent inhibits the activity of the estrogen receptor (e.g., estrogen receptor α (ERα)). Suitable anti-estrogen therapeutic agents may include but are not limited to selected estrogen receptor modulators or degraders (SERMs or SERDs). SERMs or SERDs may include, but are not limited to raloxifene or fulvestrant.

In some embodiments of the disclosed methods, a subject having or at risk for developing a hernia is administered a therapeutic agent that inhibits the production and/or activity of estrogen in the subject. In particular embodiments, the therapeutic agent inhibits the production of estrogen. Therapeutic agents that inhibit the production of estrogen may include therapeutic agents that inhibit the activity of aromatase (i.e., aromatase inhibitors). Suitable aromatase inhibitors may include, but are not limited to letrozole.

In some embodiments of the disclosed methods, a subject having or at risk for developing a hernia is administered a therapeutic agent that stimulates the production and/or increases the concentration and/or activity of androgens in the subject. In particular embodiments, the therapeutic agent is an androgen or an androgen stimulator (or an androgen analogue or derivative that binds to an androgen receptor and acts as an agonist). Suitable androgens and androgen stimulators for the disclosed methods may include, but are not limited to testosterone and analogues or derivatives thereof (e.g., 4-Androstenediol, 4-Dehydroepiandrosterone, 5-Androstenedione, 5-Dehydroandrosterone, 11β-Hydroxy-4-androstenedione, 11-Keto-4-androstenedione, 5-Androstenediol, 4-Androstenedione, 1-Methyl-δ1-4-androstenedione, δ1-4-Androstenedione, 5-Dehydroepiandrosterone, 6-Methylidene-δ1-4-androstenedione, 4-Hydroxy-4-androstenedione, and 10-Propargyl-4-androstenedione), dihydrotestosterone (DHT) and analogues or derivatives thereof (e.g., 4,5α-Dihydrotestosterone, 4,5α-Dihydro-δ1-testosterone, 11-Keto-4,5α-dihydrotestosterone, 2α-Methyl-4,5α-dihydrotestosterone, 2α,3α-Epithio-3-deketo-4,5α-dihydrotestosterone, 1α-Methyl-4,5α-dihydrotestosterone, 1-Methyl-4,5α-dihydro-δ1-testosterone, 2α-Chloro-4,5α-dihydrotestosterone 3-O-(p-nitrophenyl)oxime, and 2-Methyl-4,5α-dihydro-δ1-testosterone), 19-nortestosterone and analogues or derivatives thereof (e.g., 11β-Methyl-19-nortestosterone, 19-Nor-δ9-testosterone, 7α,11β-Dimethyl-19-nortestosterone, 4-Chloro-19-nortestosterone, 4-Hydroxy-19-nortestosterone, 19-Nor-δ9,11-testosterone, and 7α-Methyl-19-nortestosterone) 17α-Alkylated testosterone and analogues or derivatives thereof (e.g., 7β,17α-Dimethyltestosterone, 4-Chloro-17α-methyl-δ1-testosterone, 4-Hydroxy-17α-methyl-δ1-testosterone, 17α-Ethyltestosterone, 9α-Fluoro-11β-hydroxy-17α-methyltestosterone, 2-Formyl-11α-hydroxy-17α-methyl-δ1-testosterone, 17α-Methyl-2′H-androsta-2,4-dieno[3,2-c]pyrazol-17β-ol, 17α-Methyl-δ1-testosterone, 4-Chloro-17α-methyltestosterone, 17α-Methyltestosterone, 4-Hydroxy-17α-methyltestosterone, and 1α,7α-Diacetylthio-17α-methyltestosterone), 17α-Alkylated dihydrotestosterone and analogues or derivatives thereof (e.g., 3-Deketo-17α-methyl-4,5α-dihydro-δ2-testosterone, 17α-Methyl-5α-androstano[2,3-c][1,2,5]oxadiazol-17β-ol, 17α-Methyl-4,5α-dihydrotestosterone, 2α,17α-Dimethyl-4,5α-dihydrotestosterone, 17α-Methyl-4,5α-dihydro-δ1-testosterone, 3-Azi-17α-methyl-4,5α-dihydrotestosterone, 2α,3α-Epithio-3-deketo-17α-methyl-4,5α-dihydrotestosterone, 2,17α-Dimethyl-4,5α-dihydro-δ1-testosterone, 2-Oxa-17α-methyl-4,5α-dihydrotestosterone, 2-Hydroxy methylene-4,5α-dihydro-17α-methyltestosterone, and 17α-Methyl-2′H-5α-androst-2-eno[3,2-c]pyrazol-17β-ol), 17α-Alkylated 19-Dinortestosterone and analogues and derivatives thereof (e.g., 7α,17α-Dimethyl-19-nor-δ9-testosterone, 17α-Ethyl-19-nor-δ9-testosterone, 17α-Ethyl-3-deketo-19-nortestosterone, 17α-Methyl-19-nor-δ9-testosterone, 4-Hydroxy-17α-methyl-19-nortestosterone, 17α-Methyl-19-nor-δ9,11-testosterone, 7α,17α-Dimethyl-19-nortestosterone, 17α-Ethyl-18-methyl-19-nortestosterone, 17α-Ethyl-19-nortestosterone, 17α-Methyl-19-nortestosterone, 17α,18-Dimethyl-19-nor-δ9,11-testosterone, and 17α-Ethyl-18-methyl-19-nor-δ9,11-testosterone), 17α-Vinylated testosterone and analogues and derivatives thereof (e.g., 17α-Ethenyl-19-nortestosterone) 17α-Ethynylated 19-nortestosterone and analogues or derivatives thereof (e.g., 17α-Ethynyl-3-deketo-3β-hydroxy-19-nortestosterone, 17α-Ethynyl-18-methyl-19-nor-δ9,11-testosterone, (−)-17α-Ethynyl-18-methyl-19-nortestosterone, 17α-Ethynyl-3-deketo-19-nortestosterone, 17α-Ethynyl-18-methyl-19-nortestosterone, 17α-Ethynyl-19-nor-δ9,11-testosterone, and 7α-Methyl-17α-ethynyl-19-nor-δ5(10)-testosterone, R1881 (i.e., (17b)-17-Hydroxy-17-methyl-estra-4,9,11-trien-3-one, methyltrienolone, netribolone), human chorionic gonadotropin, and analogues thereof (e.g., analogues that binds to an androgen receptor as an agonists).

In the disclosed methods, a subject having or at risk for developing a hernia is administered a therapeutic agent that modulates the production and/or activity of a sex steroid hormone in the subject. In some embodiments, the therapeutic agent is administered locally to the subject at the hernia or at a site at risk for developing a hernia. The therapeutic agent may be administered in any suitable manner. In some embodiments, the therapeutic agent is administered topically at the site of the hernia or at a site that is likely to develop a hernia.

In some embodiments of the disclosed methods, the subject has undergone surgery for hernia repair or the subject is preparing to undergo surgery for hernia repair and the therapeutic agent is administered to the subject before, during, and/or after surgery. In particular embodiments, the subject has undergone surgery and the therapeutic agent is incorporated into a hernia repair mesh material that is implanted in the subject at the hernia and that releases the therapeutic agent to the hernia.

In some embodiments, the therapeutic agent is administered systemically to the subject in need of treatment or prevention for a hernia. In particular embodiments, the therapeutic agent is administered orally. In other embodiments, the therapeutic agent is administered via injection.

In other embodiments, the therapeutic agent is administered topically at site where a subject in need thereof is likely to develop a hernia. In other embodiments, the therapeutic agent is administered at a site where a subject is beginning to develop a hernia.

### ILLUSTRATIVE EMBODIMENTS

The following Embodiments are illustrative and should not be interpreted to limit the scope of the claimed subject matter.

Embodiment 1. A method for treating and/or preventing a hernia in a subject in need thereof, the method comprising administering to the subject one or more therapeutic agents that modulate the production and/or activity of one or more sex steroid hormones in the subject.

Embodiment 2. The method of embodiment 1, wherein the subject has or is at risk for developing a hernia selected from an inguinal hernia, a femoral hernia, an umbilical hernia, a hiatal hernia, an incisional hernia, and diastasis recti.

Embodiment 3. The method of embodiment 1, wherein the subject has or is at risk for developing an inguinal hernia.

Embodiment 4. The method of any of the foregoing embodiments, wherein the therapeutic agent inhibits the production and/or activity of estrogen.

Embodiment 5. The method of embodiment 4, wherein the therapeutic agent is an anti-estrogen therapeutic that inhibits the activity of estrogen in the subject.

Embodiment 6. The method of embodiment 5, wherein the anti-estrogen therapeutic is a selective estrogen receptor modulator or degrader (SERM or SERD).

Embodiment 7. The method of embodiment 6, wherein the SERM or SERD is raloxifene or fulvestrant.

Embodiment 8. The method of embodiment 4, wherein the therapeutic agent inhibits the production of estrogen in the subject.

Embodiment 9. The method of embodiment 8, wherein the therapeutic agent is an aromatase inhibitor.

Embodiment 10. The method of embodiment 9, wherein the aromatase inhibitor is letrozole.

Embodiment 11. The method of any of embodiments 1-3, wherein the therapeutic agent stimulates the production and/or activity of androgens in the subject.

Embodiment 12. The method of embodiment 11, wherein the therapeutic agent is an androgen or an androgen stimulator.

Embodiment 13. The method of embodiment 12, wherein the androgen or androgen stimulator is testosterone, dihydrotestosterone, 19-Nortestosterone, 17α-alkylated testostereon, 17α-alkylated dihydrotestosteron, 17α-alkylated 19-nortestosterone, 17α-vinylated testosterone, 17α-ethynylated testosterone, R1881 (i.e., (17b)-17-Hydroxy-17-methyl-Estra-4,9,11-trien-3-one, Methyltrienolone, Metribolone), human chorionic gonadotropin, or an analogue or derivative thereof (e.g., an analogue or derivative that binds to an androgen receipt as an agonist.

Embodiment 14. The method of any of the foregoing embodiments, wherein the therapeutic agent is administered locally to the subject at the hernia or at a site at risk for developing a hernia.

Embodiment 15. The method of any of the foregoing embodiment, wherein the therapeutic agent is administered topically.

Embodiment 16. The method of any of the foregoing embodiments, wherein the subject has undergone surgery for hernia repair or the subject is preparing to undergo surgery for hernia repair and the therapeutic agent is administered to the subject before, during, and/or after surgery.

Embodiment 17. The method of any of the foregoing embodiments, wherein the subject has undergone surgery and the therapeutic agent is incorporated into a hernia repair mesh material that is implanted in the subject at the hernia and that releases the therapeutic agent to the hernia.

Embodiment 18. The method of any of the foregoing embodiments, wherein the therapeutic agent is administered systemically.

Embodiment 19. The method of any of the foregoing embodiment, wherein the therapeutic agent is administered orally.

Embodiment 20. The method of any of the foregoing embodiments, wherein the therapeutic agent is administered via injection.

### EXAMPLES

The following Examples are illustrative and should not be interpreted to limit the scope of the claimed subject matter.

### Example 1—Specific Aims and Research Strategy

More than 1 in 4 men can expect to develop symptomatic inguinal hernia, and in the US, over 600,000 inguinal hernia repair surgeries are performed annually. Despite its prevalence, the biologic or genetic basis of inguinal hernia is unknown. Refractory hernias accompanied by high mortality, high recurrence, and long-term severe pain continue to challenge surgeons and patients. The long-term objective of this application is to determine the role of estrogen action in the etiology of lower abdominal muscle tissue (LAMT) fibrosis and atrophy associated with a subset of inguinal hernias. Testosterone in males is converted by aromatase to estradiol (E2) in target tissues, which exerts estrogenic actions via estrogen receptor (ER). Aromatase is expressed only in the brain, testes, and gonadal fat of male mice, whereas in men, aromatase is expressed in many other tissues (muscle, fat) to provide physiologically necessary levels of estrogen. We generated two transgenic humanized aromatase (Arom^{hum}) mouse lines, each containing a single copy of the entire human aromatase gene including its regulatory region, to mimic human physiology for aromatase expression and estrogen production.

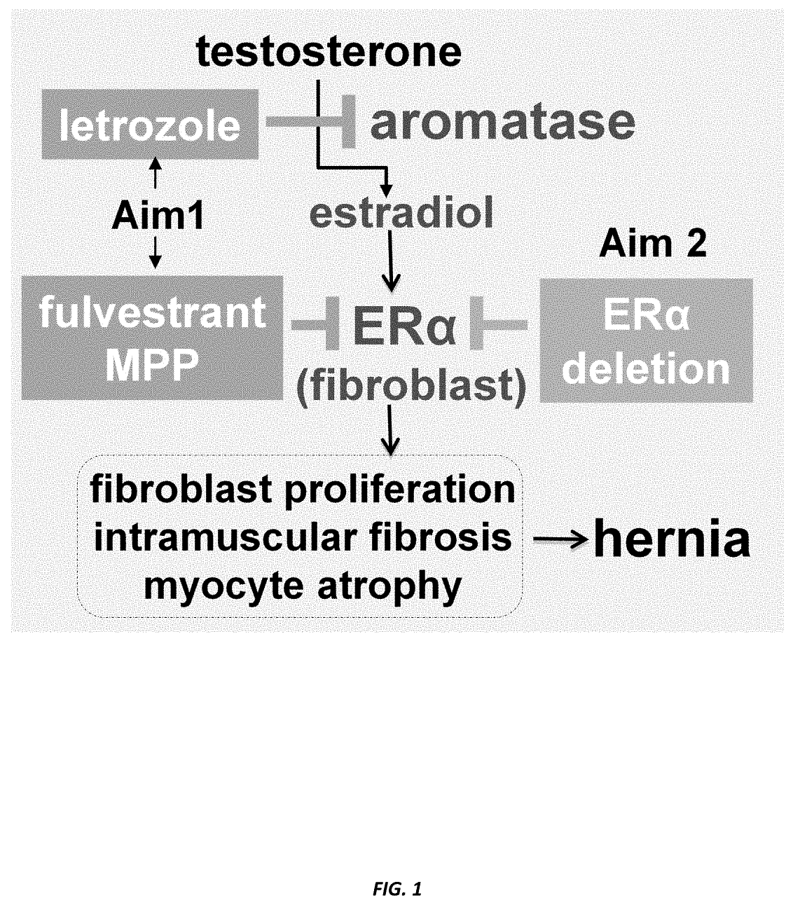

The LAMT stromal component contains the largest populations of ERα-expressing fibroblasts and is extremely sensitive to local aromatase activity and estrogen formation. We found that local E2 formation arising from physiologic levels of expression of the human aromatase gene in mouse LAMT was associated with LAMT fibrosis and weakness characterized by progressive replacement of atrophic myocytes with ERα-rich fibroblasts and formation of large inguinal hernias in >90% of Arom^{hum} male mice. Postnatal administration of an aromatase inhibitor entirely prevented this phenotype. No hernias were observed in any of the wild-type (WT) littermates. LAMT showed activated profibrotic pathways and enhanced estrogen action in Arom^{hum} vs. WT mice. We hypothesize that enhanced estrogen action caused by locally formed E2, drives muscle fibrosis and atrophy, leading to the hernia phenotype affecting highly estrogen-sensitive portions of skeletal muscle tissue, which is LAMT in Arom^{hum} mice. Our hypothesis is consistent with the remarkable and parallel increases in skeletal muscle atrophy, inguinal hernia incidence, and aromatase expression in the skeletal muscle and other peripheral tissues in a subset of aging men. We propose the following aims (FIG. 1):

Aim 1. Determine the mechanism for LAMT fibrosis, myocyte atrophy, and hernia formation involving aromatase, E2 and ERα. Hypothesis: E2 produced as a result of aromatase expression in estrogen-sensitive LAMT stimulates disordered proliferation of ERα-expressing fibroblasts and production of extracellular matrix (ECM), which progressively replaces atrophic myocytes, leading to LAMT weakness and hernia development. Treatment with an aromatase inhibitor, letrozole, completely prevented this phenotype. Experiments 1a: We will further define whether this phenotype can be prevented with early administration of the potent E2 antagonist, fulvestrant, or the highly ERα-selective E2 antagonist Methyl-Piperidino-Pyrazole (MPP) in Arom^{hum} mice. We will also determine whether the combined treatment of Arom^{hum} mice with fulvestrant, MPP, and/or letrozole can arrest or reverse LAMT fibrosis, myocyte atrophy, or small hernias in early developmental stages. Experiments 1b: We will determine the genome-wide ERα cistrome, histone modification maps, and transcriptomes in LAMT fibroblasts of Arom^{hum} mice employing ERα-ChIP-seq and RNA-seq in the presence or absence of E2 or fulvestrant. Functional ERα targets will be assessed by integrative bioinformatics and pathway analyses and verified by real-time PCR, immunohistochemistry and immunoblotting.

Aim 2. Determine whether conditional knockout of ERα in fibroblasts diminishes muscle fibrosis, myocyte atrophy or hernia and associate the mechanistic data from mouse experiments with human disease. Hypothesis: ERα, expressed in strikingly high levels in LAMT fibroblasts, is responsible for mediating the effects of E2, causing LAMT fibroblast proliferation, ECM formation, myocyte atrophy, and hernia formation. Experiments 2a: To selectively ablate ERα in the fibroblast component of LAMT, we will cross Arom^{hum} mice with mice bearing selective ERα (Esr1) disruption in fibroblasts using the fibroblast-specific-protein-1-Cre (Fsp1-Cre; Esr1^{fl/fl}). Diminished fibrosis, muscle atrophy, and hernia formation in Arom^{hum} mice with fibroblast-specific disruption of ERα will provide conclusive evidence for the role of E2/ERα signaling in causing muscle weakness and hernia formation. Experiments 2b: We will associate the mechanistic data with human inguinal hernia. Using hernia wall/LAMT biopsies from men with or without hernia, we will employ morphometry to determine the proportions and size of myocytes and area of fibrosis, and assess tissue E2 levels, aromatase and ERα mRNA/protein, and estrogen-target and profibrotic gene signatures.

Here, we propose studies to define the underlying mechanism for a previously unrecognized role of estrogen signaling in muscle fibrosis and atrophy in the lower abdominal wall using the first and only mechanistic model for inguinal hernia. Definition of molecular pathways responsible for muscle fibrosis and weakening will likely lead to new interventional strategies to potentially prevent hernia development in high-risk populations and provide adjuvant treatment options to surgical repair of refractory abdominal hernias in a subset of elderly men.

A. Significance

Inguinal hernia repair is the most common general surgical procedure performed in the US today (600,000/year)¹⁻³. More than 1 in 4 men will undergo inguinal hernia repair during their lifetime³⁻⁵. Annual health care costs directly attributable to inguinal hernia exceed $2.5 billion in the US⁶. Although surgery can cure the majority of inguinal hernias in otherwise healthy patients, recurrent hernias in very elderly men or men with severe diabetes and liver failure challenge surgeons due to high morbidity and mortality and high rate of complications (e.g., wound infection, recurrence, and long-term severe pain)⁷⁻¹³. Despite its extraordinarily high prevalence and the rate of recurrence, the etiology, pathophysiology, and pharmacological treatment strategies of inguinal hernia are severely understudied; the RePORTER database does not contain a single funded grant investigating this topic.

Lower abdominal muscle tissue (LAMT) where hernias occur is composed of layers of oblique and transverse skeletal muscle made of myofibers (myocytes) and stromal tissue, a mixture of well-organized fibroblasts and extracellular matrix (ECM). Stromal tissue surrounds these muscle groups, fascicles or myocytes and eventually forms the deep fascia as an extension of muscle. Based on our preliminary findings, we hypothesize that age-related muscle atrophy may occur as an estrogen-induced replacement of myofibers by estrogen-induced fibroblast proliferation in a subset of elderly men predisposed to develop hernias involving LAMT and including inguinal hernias¹⁴⁻¹⁹. Since the estrogen-induced inguinal hernias in our mouse model and also in elderly men were conclusively linked to myofiber atrophy and fibrosis in LAMT, we envision that certain subsets of both indirect or direct inguinal hernias in elderly men or men with severe chronic illness may be related to this mechanism^{6, 7, 20-25,26}. The high prevalence of inguinal hernias, combined with poor surgical outcomes in recurrent hernias and high associated costs, reveals a significant need for developing preventive therapeutic strategies in high-risk populations.

The mechanisms of inguinal hernia development in elderly men is possibly multifactorial and severely understudied. Most research to date focuses on the biophysical pressures now known to play a role in hernia development even though risk factors such as diabetes, steroid use, or genetic predisposition were reported²⁷. Age is also a known significant risk factor: hernia incidence peaks between 60 and 75 years of age, with an approximately 50% annual incidence in men by the age of 75²⁸⁻³⁰. Aromatase is the key enzyme for estrogen biosynthesis via conversion of testosterone (T) to estradiol (E2) in men^{31, 32} Aromatase expression and enzyme activity in peripheral human tissues such as skeletal muscle and fat increase with advancing age up to 4-fold^{32, 33} In parallel, the tissue levels of the biologically potent estrogen, E2, in men significantly increase with aging and become prominent after age 40^{34, 35}. Experimentally, exogenous administration of estrogen to mice or local production of estrogen in skeletal muscle in a humanized aromatase mouse model (Arom^{hum}, see below) causes LAMT atrophy and inguinal hernia, similar to inguinal hernia in a subset of elderly men³⁶⁻³⁸. Previously, mice models served as common antecedents to clinical trials for the study of hernia development and response to repair techniques; these results were directly translated to human models³⁹⁻⁴¹. We posit that the age-related shift in sex steroid hormones, with increased skeletal muscle tissue E2 levels may disproportionately affect highly estrogen-sensitive sites such as LAMT and be associated with inguinal hernia development in in a subset of elderly men. A better understanding of this mechanism may reveal new adjuvant therapeutic approaches. We will thus explore these mechanisms in mice and then sample human tissue in order to determine whether similar hormonal level variation exist in men with and without hernias setting us up for future studies in humans^{42, 43}.

Scientific premise. Strengths. In the 1930s, two separate laboratories reported that approximately 40% of male mice that received estrogen injections or ovarian grafts as early as postnatal day 28 or as late as 30 weeks of age developed inguinal hernias³⁶⁻³⁸. This suggested a causal link between estrogen and hernia formation, but this model was not physiologically relevant or amenable to mechanistic manipulation. Our novel physiologically relevant humanized mouse model containing the entire human aromatase gene (Arom^{hum}) provide the first set of mechanistic studies of the link between estrogen formation, muscle fibrosis and atrophy, and hernia analogous to human disease. The phenotype is very clear-cut; 90-100% of male Arom^{hum} mice develop LAMT muscle atrophy, fibrosis, and hernia by 24 weeks of age; an aromatase inhibitor started at 3 weeks entirely prevents the phenotype. This fibrotic mechanism is mediated by estrogen receptor α (ERα located in the fibroblast component of the skeletal muscle tissue and activated by E2 produced by local aromatization of testosterone (T). Thus, the critical pathological process is estrogen-driven. These mice also have decreased circulating T and increased muscle tissue E2 levels, a hormonal profile that resonate with a subset of elderly men who are prone to hernia development. Weaknesses. The timing of aromatase/estrogen excess in muscle tissue and hernia development in male Arom^{hum} mice and elderly men are different. Arom^{hum} mice start developing hernia at puberty with the first increases in circulating T, the substrate for muscle aromatase and precursor for E2. It takes decades in men to reach a similar hormonal profile and hernia development. Arom^{hum} mice have lower T levels compared with WT mice, which is consistent with low T and high E2 levels in elderly men. This, however, raises the question of potential contributing roles of low T and possibly androgen receptor in hernia development, which are out of scope for this application and will be studied in the future.

B. Innovation

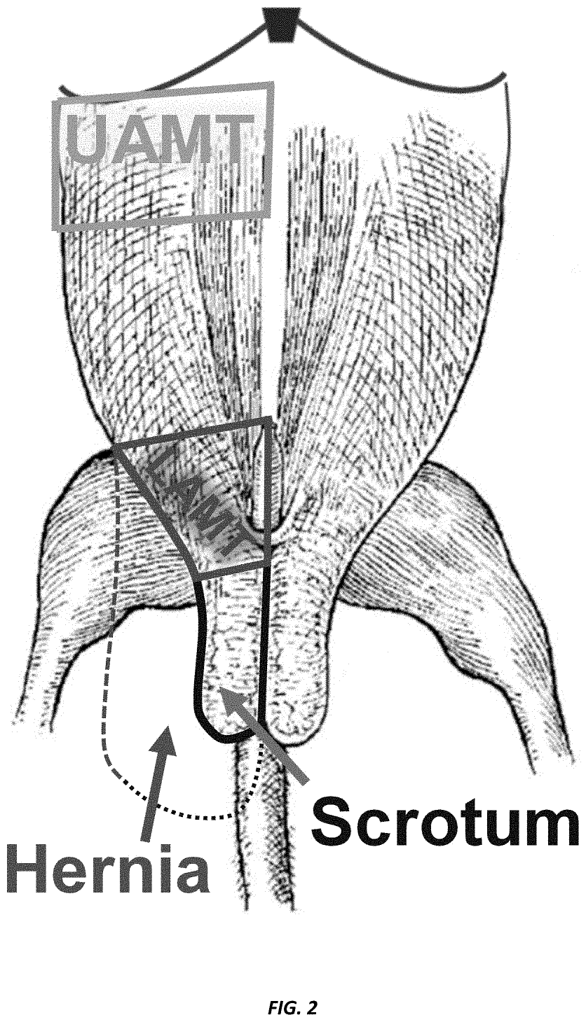

In wild-type (WT) male mice, aromatase is expressed in the brain and testes and is barely detectable in gonadal fat, whereas men express aromatase in many peripheral tissues, including fat and skeletal muscle^{32, 44}. We generated a unique tool, the Arom^{hum} transgenic mouse model, the phenotype of which imitates the pattern of estrogen production in the tissues of a subset of elderly men. Arom^{hum} mice contain the human CYP19A1 (aromatase) gene with its 5′-regulatory region, and nearly all male Arom^{hum} mice spontaneously develop inguinal hernias, which resonate high hernia incidence in elderly men. This model is amenable to drug development because postnatal treatment with an aromatase inhibitor entirely prevents hernia development. We will also test the following novel concepts. Increased muscle tissue expression of aromatase and E2 levels seem to drive the phenotype both in Arom^{hum} mice and elderly men as opposed to increased circulating E2 levels. In contrast to upper abdominal muscle tissue (UAMT), LAMT contains strikingly higher ERα levels in its stromal fibroblast component and thus uniquely E2-sensitive (FIG. 2). Triggered by increased LAMT E2 levels, ERα-rich fibroblasts proliferate and initiate fibrosis leading to myocyte atrophy, overall muscle weakness and eventually inguinal hernia phenotype in Arom^{hum} mice. Neither the hypothesis linking estrogen to inguinal hernia development nor the unique Arom^{hum} mouse model of inguinal hernia has been studied and are therefore novel.

C. Approach

Overall hypothesis. Increased estrogen action is instrumental for LAMT stromal fibrosis, muscle atrophy, and hernia formation. In particular, disordered fibroblast proliferation and increased ECM formation in the fascial component causes weakening of LAMT and herniation of abdominal contents into the scrotum.

Overall strategy. Under Aim 1, we will determine the roles of excess E2 formation and high ERα levels in LAMT leading to disordered fibroblast proliferation, excessive ECM production, myocyte atrophy, and inguinal hernia formation in the Arom^{hum} mouse model. We will perform ERα-ChIP-seq and RNA-seq on primary LAMT fibroblasts of Arom^{hum} in the presence of E2 or its antagonist to define genome-wide estrogen target genes. ERα target gene signature will be assessed by integrative bioinformatics, pathway analysis, real-time PCR, immunohistochemistry (IHC), and immunoblotting. Under Aim 2, we will define the underlying molecular mechanisms of hernia formation via genetic disruption of ERα selectively in fibroblasts in mice and associate the mechanistic data from mouse experiments with human disease. The majority of the experiments will employ whole tissues or isolated primary fibroblasts from LAMT or UAMT of 4-week-old (before hernia formation) or 24-week-old Arom^{hum} male mice (after hernia formation). Sampling sites for UAMT, healthy LAMT or fibrotic LAMT that comprises the hernia wall are shown in FIG. 2. To determine the role of E2 in hernia formation, we will use several different strategies, including extended-release pellets of letrozole (aromatase inhibitor), IC1182270 (fulvestrant, general E2 antagonist), and Methyl-Piperidino-Pyrazole (MPP, ERα-selective E2 antagonist). We will recapitulate these mechanistic findings in fibrotic and healthy LAMTs from men with or without hernia.

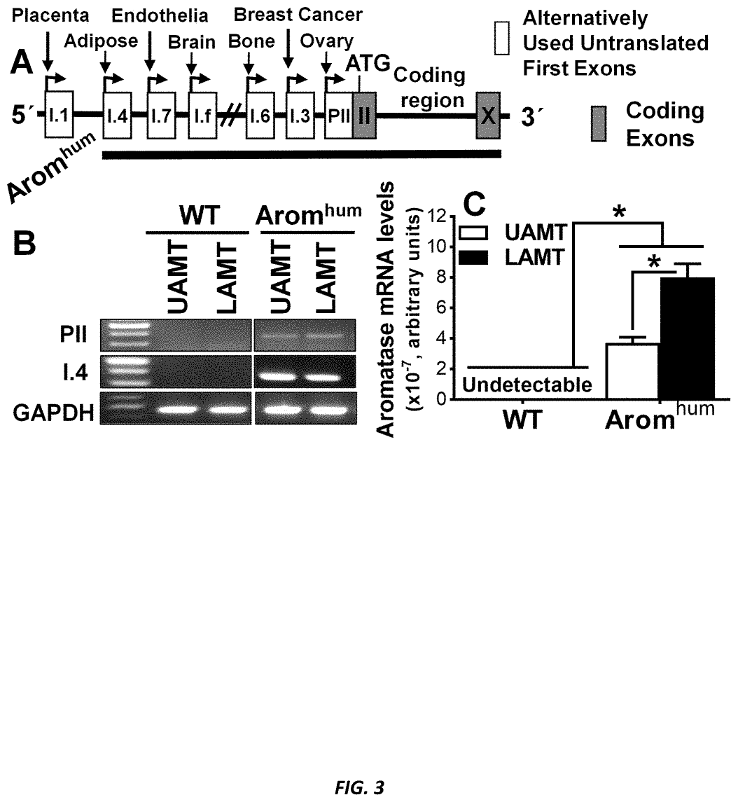

Scientific Rigor. We present compelling and reproducible preliminary evidence generated using a mouse model (Arom^{hum}) that is physiologically relevant to human disease in terms of the estrogen profile (FIG. 3). Our initial observations of inguinal hernias in male mice several years ago were coincidental and thus unbiased because at the time, we were focused on studying breast hyperplasia in female mice⁴⁵. Since then, the inguinal hernia phenotype has been reproducibly observed in 90-100% of all of the three Arom^{hum} male mouse lines independently by several lab members. No females developed hernias. The observation that an aromatase inhibitor prevented hernia in all treated Arom^{hum} mice added further rigor. Our study design is robust, utilizing Arom^{hum} mice with endpoints examined before and after hernia formation, and compared to those of WT littermates using 2 different tissue sites: UAMT and LAMT (normal or weakened/herniated). Inguinal hernia affects predominantly men vs women (10:1 ratio). Inguinal hernias are unique to male mice and aged men. Here, we will focus on the effects of estrogen action on skeletal muscle integrity and hernia formation. Thus, our work will be carried out primarily in male mice or tissues primarily from men over 40 years of age. We will use mouse or human tissues or primary cells, which closely represent in vivo pathology.

Statistical Methods. Statistical analyses will be performed with the help of the Biostatistics Core Facility of Northwestern University. Normality of biological data distribution will be checked and log-transformations made for non-normal data. These variables will be compared across different groups of mice for each aim using Kaplan-Meier curves, t-test or ANOVA followed by Tukey (parametric) or Chi-square tests of independence (non-parametric)⁴⁶. Sample size calculations are based on the published results of similar studies from our laboratory^{44, 45, 47-50}. For most experiments, 15 mice in each treatment group at each time point are proposed to be adequate to detect at least 30% difference, at 80% power in physiological and morphometric parameters, assuming a 30% coefficient of variation. Bioinformatics analysis for ChIP-seq, RNA-seq, and their integration is discussed in detail under Hypothesis 1b. All surgical procedures, substance injections, sample collections, and histological analyses will be performed in a double-blinded fashion. If a larger sample size appears to be necessary, the experiments will be repeated under the same conditions. Statistical significance will be assigned if two-tailed p<0.05.

Aim 1. Determine the mechanism for LAMT fibrosis, myocyte atrophy, and hernia formation involving aromatase, E2 and ERα.

Hypothesis 1a. E2 produced via aromatase expression in estrogen-sensitive LAMT stimulates disordered proliferation of ERα-expressing stromal fibroblasts and increased production of ECM, which progressively replace the myofiber component of muscle tissue, leading to LAMT myocyte atrophy, weakness and hernia.

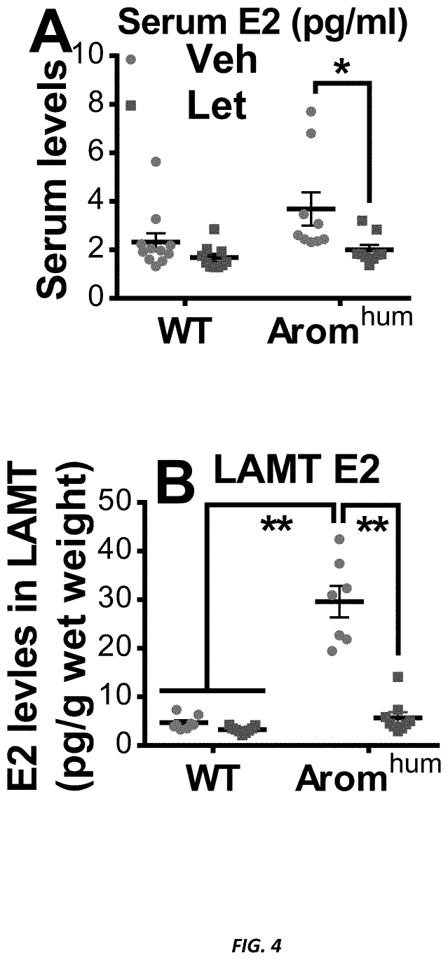

Rationale. Understanding the mechanistic link between estrogen production in LAMT, differential ERα expression, myocyte atrophy, and extensive muscle fibrosis will provide critical evidence supporting a causal link between estrogen action and the development of inguinal hernias in men. We provide evidence for differential tissue expression of ERα or aromatase that regulate estrogen action in WT and Arom^{hum} mice. In LAMT and UAMT of mice, ERα was localized to the nuclei of fibroblasts in epimysial, endomysial or perimysial stromal tissue, which is collectively referred to as fascia. In both in WT and Arom^{hum} mice, LAMT contained higher ERα levels than UAMT. Mouse or human aromatase expression was absent in WT tissue, whereas human aromatase expression was readily detectable in all Arom^{hum} skeletal muscle tissues including LAMT and UAMT (FIG. 3). Consequently, local LAMT E2 levels were higher in WT than Arom^{hum} mice (FIG. 4). Thus, elevated local E2 production in skeletal muscle tissue, via aromatization of T, appear to be the key factor in extensive LAMT fibrosis, myocyte atrophy, and hernia formation.

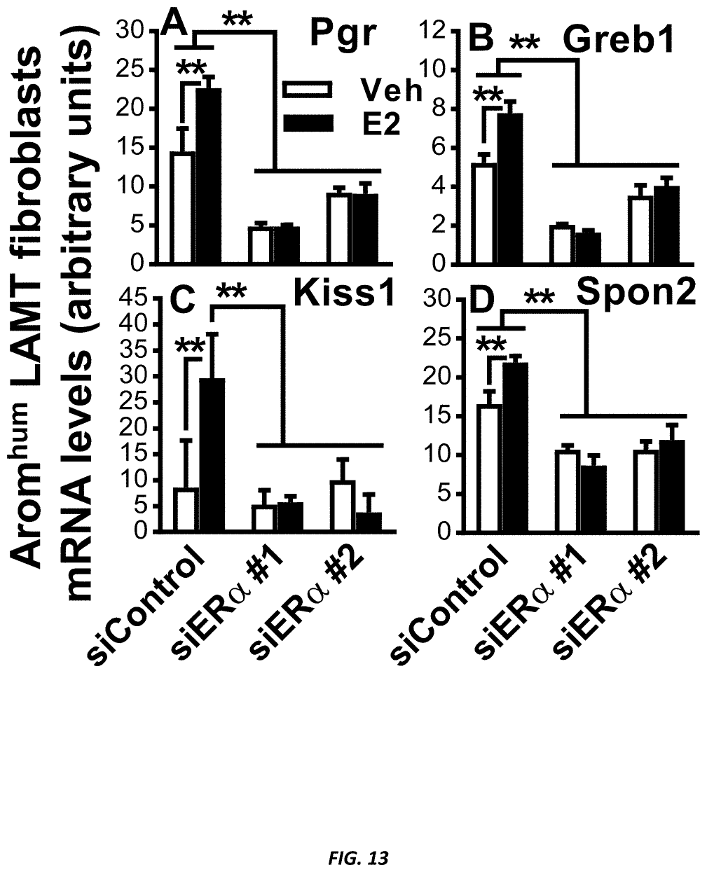

The LAMT of WT or Arom^{hum} male mice, compared with UAMT or other skeletal muscles, including quadriceps muscle tissues, contains higher quantities of perimysial and endomysial stromal tissue that is rich in ERα-expressing fibroblasts (see FIG. 13 below). Although this feature of LAMT renders it more sensitive to estrogen, this does not lead to a phenotypic consequence in WT mice, since mouse peripheral tissues such as muscle tissue do not express aromatase or produce E2. In contrast, LAMT in Arom^{hum} mice contains strikingly higher levels of aromatase and locally produced E2 (FIGS. 3 and 4). However, circulating E2 levels are similar in WT and Arom^{hum} mice. Additionally, mRNA levels of ERα in LAMT fibroblasts of Arom^{hum} mice are 1786- and 215-fold higher than those of the other estrogen receptor subtypes, ERβ and Gpr30, respectively (data not shown), indicating ERα seems to be the predominant receptor that plays a key role in estrogen sensitivity and hernia formation. Thus, locally produced E2 in LAMT, but not circulating E2, is strongly linked to disordered proliferation of ERα-expressing fibroblasts, excessive ECM formation and myocyte atrophy, thus weakening both fascial and myofiber components of skeletal muscle tissue leading to herniation of the abdominal contents.

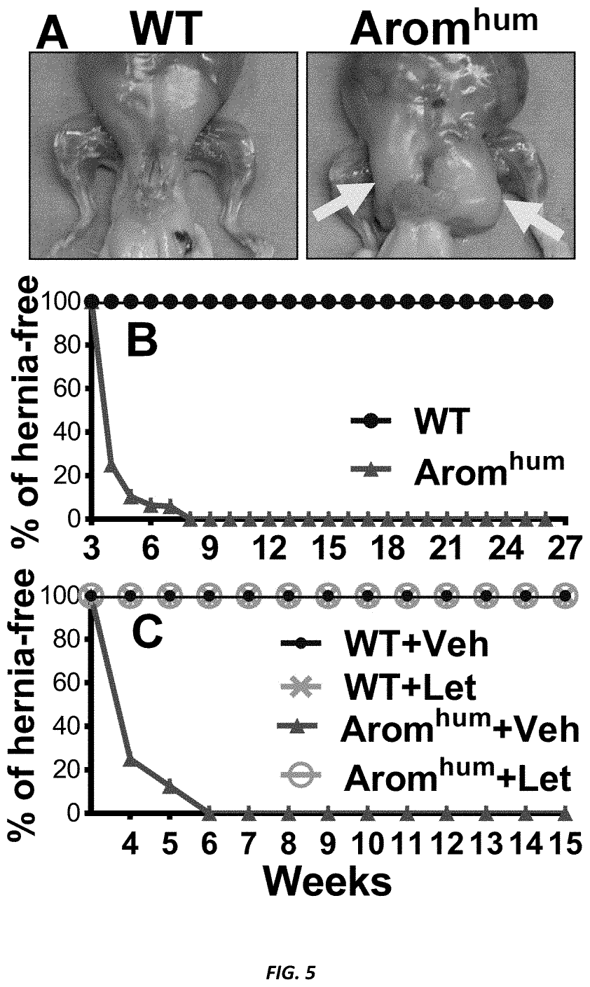

E2 is the biologically active estrogen that is necessary for bone mineralization and maintenance of lipid homeostasis in both men and women^{31, 51}. E2 also induces fibrosis in pathologic tissues including gynecomastia, scleroderma, and uterine fibroid tumors⁵²⁻⁵⁴. The physiologic or pathologic roles of E2 in skeletal muscle tissue, however, are not well understood. Treatment of Arom^{hum} mice with the aromatase inhibitor letrozole starting at 3 weeks of age entirely prevented LAMT fibrosis or inguinal hernia development in all animals (FIG. 5). Our data suggest that local conversion of T to E2 via aromatase within LAMT of Arom^{hum} mice accounts for the ready availability of estrogen to LAMT, which is associated with hernia formation.

In men, conversion of circulating T to E2 via aromatase expression in bulky tissues, namely, skeletal muscle and adipose tissue, produces the majority of estrogen⁵⁵⁻⁵⁷. Muscle and adipose tissue aromatase expression increases with advancing age by up to 4-fold and peaks after age 60, which coincides with the peak incidence of inguinal hernia⁵⁷⁻⁵⁹. Notably, hernias in Arom^{hum} mice are first observed at 4 weeks, immediately after the start of secretion of testicular T, which is the substrate for muscle aromatase. Moreover, based on the circulating levels of T and E2 and estimated metabolic clearance rates, we calculated that blood-to-blood peripheral conversion of T to E2 in male Arom^{hum} mice is about 0.3%, which is similar to that observed in elderly men who represent the highest risk group for developing hernia⁵⁷⁻⁶⁹. Arom^{hum} mice thus represent a unique and pathologically relevant model available to study the relationship between hernia development and the aromatization of T to E2 and downstream estrogenic effects in skeletal muscle. Here, we will utilize the Arom^{hum} mouse model to define the roles of aromatase, excess E2 in LAMT and ERα-mediated estrogen action (in fibroblasts) in LAMT fibrosis, atrophy, and hernia formation (FIG. 5).

Preliminary Data.

Human aromatase expression is higher in LAMT of Arom^{hum} mice. We found that male WT (FVB/N, Harlan) mice express aromatase only in the brain, testes and attached gonadal fat pad; the latter contains barely detectable aromatase expression^{44, 45}. In contrast, men express significant quantities of aromatase in subcutaneous fat and skeletal muscle^{55, 61}. In order to humanize mice with respect to estrogen production, we injected a linearized BAC clone containing the human aromatase gene into pronuclear FVB/N (Harlan) mouse embryos and generated three transgenic mouse lines that harbor the full (29 kb) coding region and its 3′-end (FIG. 3A)^{44, 45}. These lines also contain the 5′-flanking region encompassing alternatively used promoters of the human gene in fat, skin, bone, muscle, endothelium, brain, bone marrow, and the ovary^{32, 62}. Hernia phenotype was noted and quantified as 90-100% in all lines. We proceeded our experiments using the line represented in FIGS. 3-10. The tissue-selective promoters provide the regulatory signature for aromatase expression in each tissue^{44,45}. Regardless of promoter usage, the translated aromatase protein and its enzyme activity are identical^{44,45}. Male Arom^{hum} mice express high levels of aromatase in LAMT and UAMT via promoters 1.4 and PII (FIG. 3B, C), which is similar to human skeletal muscle tissue^{55, 61}. The genotype and phenotype are transmitted in an autosomal dominant manner (50% penetrance) by the single copy of the transgene^{44,45}. Both sexes are fertile^{44, 45}.

LAMT estrogen levels are increased in Arom^{hum} mice. Arom^{hum} mice recapitulate the primary aspects of aromatase expression in men in that most peripheral (extragonadal) tissues, such as skeletal muscle and adipose tissue, express aromatase primarily using the distal promoter 1.4 and, to a lesser extent, the proximal promoter II (FIG. 3)³². Despite additional aromatase expression from the human gene in peripheral tissues (e.g., muscle), circulating E2 levels in the Arom^{hum} mouse lines are not significantly higher than those of WT littermates measured by liquid chromatography-tandem mass spectrometry (LS-MS²) (FIG. 4A). Local E2 levels in LAMT, however, are strikingly higher in Arom^{hum} mice than in WT mice (FIG. 4B). These results suggest that locally produced E2 in muscle tissue, but not systemic E2 levels, may be the primary cause for the development of inguinal hernia phenotype in Arom^{hum} mice.

Increased LAMT estrogen levels are associated with LAMT fibrosis, myocyte atrophy, and inguinal hernia development; aromatase inhibitor prevents hernia formation in Arom^{hum} mice. Lower abdominal bulging was observed in 75% of Arom^{hum} male mice at 4 weeks; by 8 weeks, bulging was noticeable in all Arom^{hum} male mice (n=29); and by 12 weeks, all animals showed evidence of obvious inguinal hernia (FIG. 5A, B). None of the WT littermates (n=32) developed hernia. Another closely studied Arom^{hum} line also contained high levels of aromatase mRNA and E2 levels in skeletal muscle tissues including LAMT and demonstrated the phenotype of LAMT fibrosis, myocyte atrophy and hernia in 90% of male mice (data not shown, n=17). The development of hernia was positively associated with aromatase activity because administration of letrozole (a specific aromatase inhibitor, 10 μg daily via subcutaneous slow-release pellets) starting at week 3 restored LAMT E2 levels similar to WT LAMT levels (FIG. 4) and prevented hernia development in all Arom^{hum} mice (FIG. 5C).

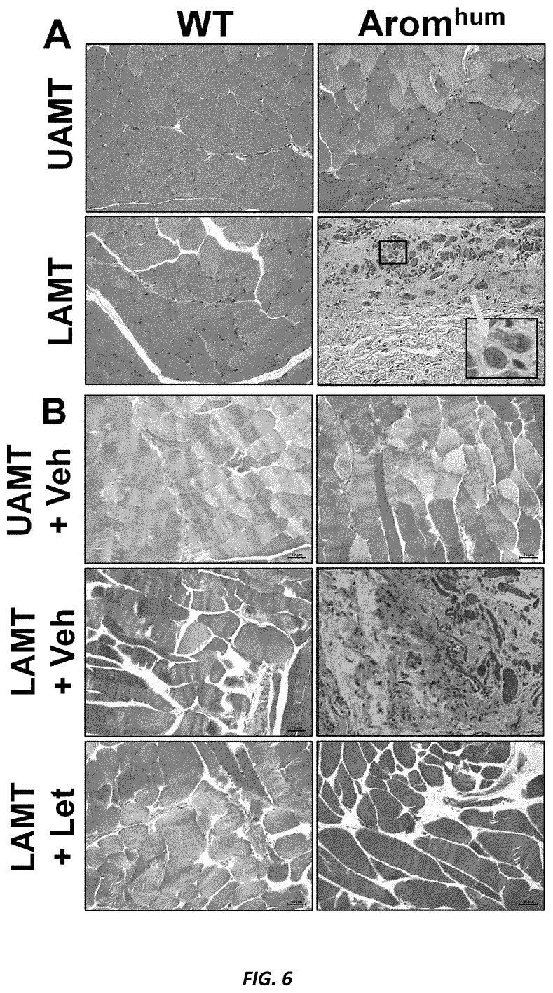

In the mouse, the scrotum is connected to the peritoneal cavity with a narrow funicular (or inguinal) canal, whose walls are composed of LAMT. Structurally normal LAMT is essential to maintain the tone of this canal and prevents herniation of bowel. LAMT is the critical site affected by E2 excess (FIGS. 2, 5, 6). As in many other tissues, stromal cells intimately interact with parenchymal cells (myocytes) in UAMT and LAMT (FIG. 6). Histological examination by H&E and Masson's trichrome staining (MTS) at 24 weeks demonstrated that LAMT but not UAMT in Arom^{hum} mice exhibited increased and disordered stromal fibroblasts, ECM formation (fibrosis) and reduced myofiber size with centrally located nuclei, indicating accelerated muscle atrophy and insufficient regeneration activity with a net result of muscle loss (yellow arrow/insert; FIG. 6A, B). Administration of the aromatase inhibitor letrozole started at 3 weeks of age for 12 weeks prevented LAMT atrophy, fibrosis and herniation in all (10 out of 10) treated Arom^{hum} mice (FIG. 6B). These observations suggest that excessive local E2 may weaken inguinal LAMT via muscle fibrosis and atrophy, which significantly enlarged the caliber of the inguinal passageway that gave way to herniation of abdominal contents into the scrotum (FIG. 2).

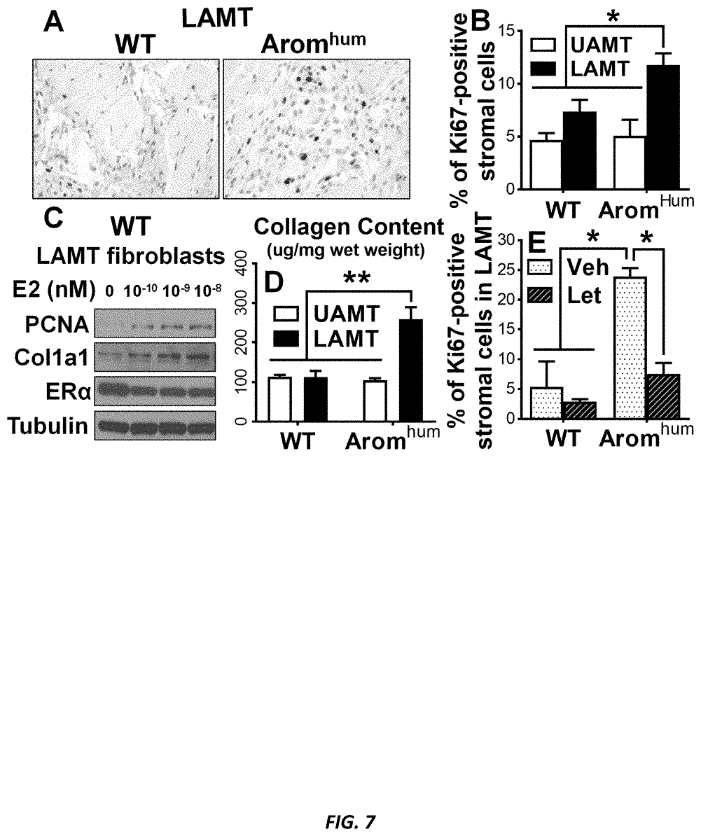

Estrogen induces disordered fibroblast proliferation and ECM formation in LAMT. We observed diffuse and disordered fibroblast proliferation and increased ECM in perimysial and endomysial stromal components of LAMT of Arom^{hum} mice (FIG. 6B). Stromal cell proliferation identified by elevated immunoreactivity for Ki67 was consistently observed in LAMT of Arom^{hum} mice compared with WT mice (FIG. 7A, B). Primary LAMT fibroblasts from WT mice treated with doses of E2 (10⁻⁸-10¹⁰ M) comparable to that seen in Arom^{hum} LAMT for 24 h showed increased proliferative activity evident by PCNA expression (FIG. 7A-C). E2 stimulated ECM deposition as shown by increased type I collagen (Col1a1) expression (FIG. 7C). The total collagen content in Arom^{hum} LAMT was higher than Arom^{hum} UAMT, WT LAMT or WT UAMT (FIG. 7D). Letrozole treatment significantly decreased stromal cell proliferative activity (FIG. 7E) and ECM content (FIG. 7B) in LAMT of Arom^{hum} mice.

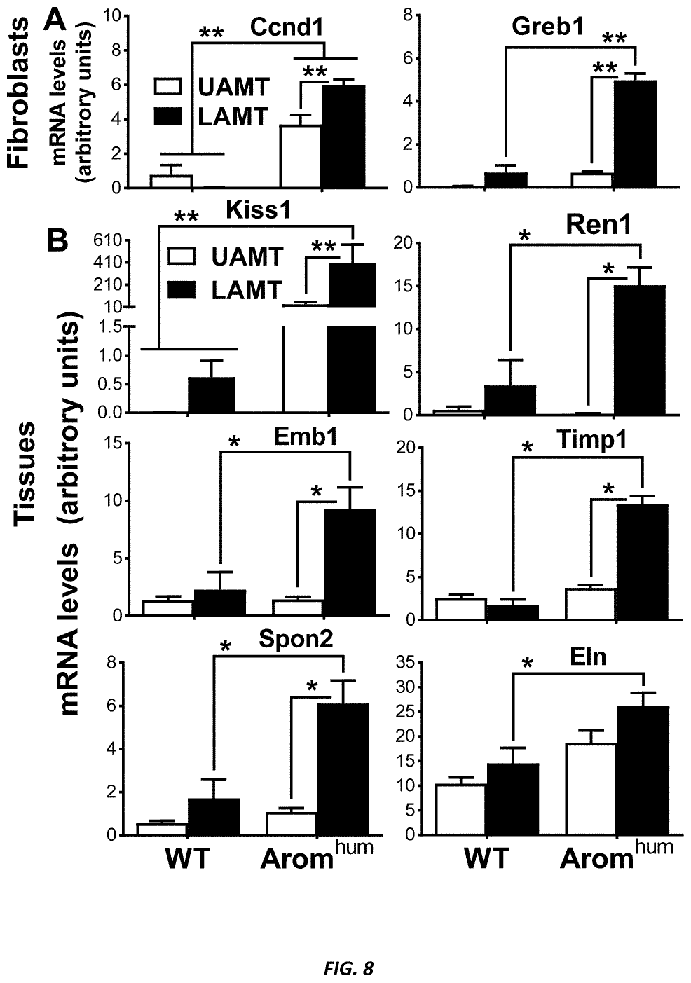

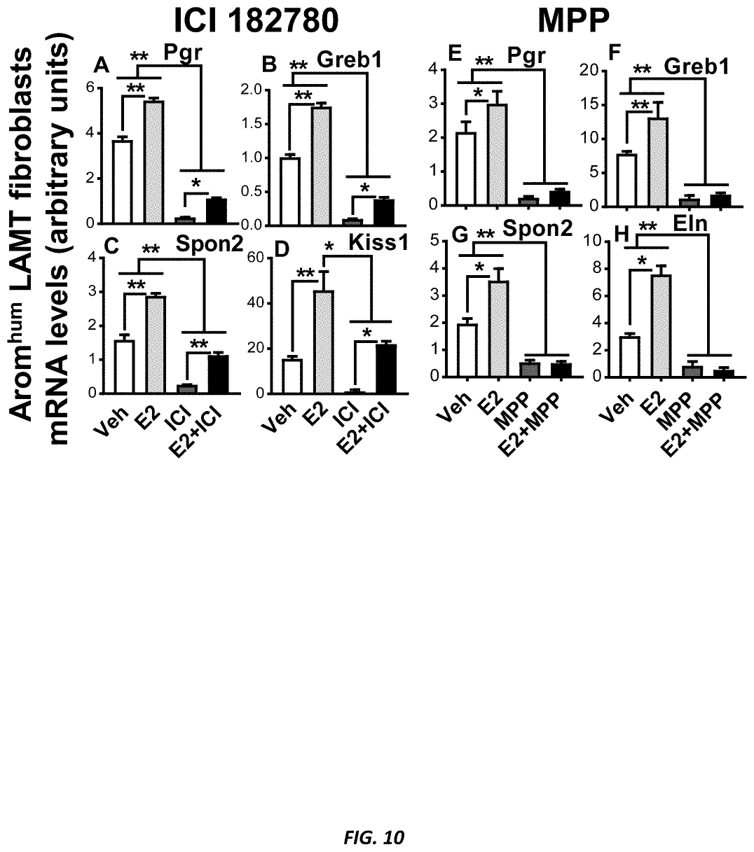

A microarray gene profiling of total RNA from LAMT of WT vs. Arom^{hum} mice (n=4/group) at 3 weeks of age (immediately before hernia development) showed that mRNA levels of estrogen-target (Ccnd1, Greb1, and Pgr) and fibrosis-related genes (Kiss1, Ren1, Emb, Timp1, Spon2, and Eln) were significantly increased in Arom^{hum} LAMT vs WT LAMT. Differential expression was verified by real-time quantitative RT-PCR (qPCR; see FIGS. 8 and 10). We concluded that ready availability of the ligand E2 prompts ERα-rich fibroblast proliferation and ECM accumulation in LAMT of Arom^{hum} mice, leading to hernia formation.

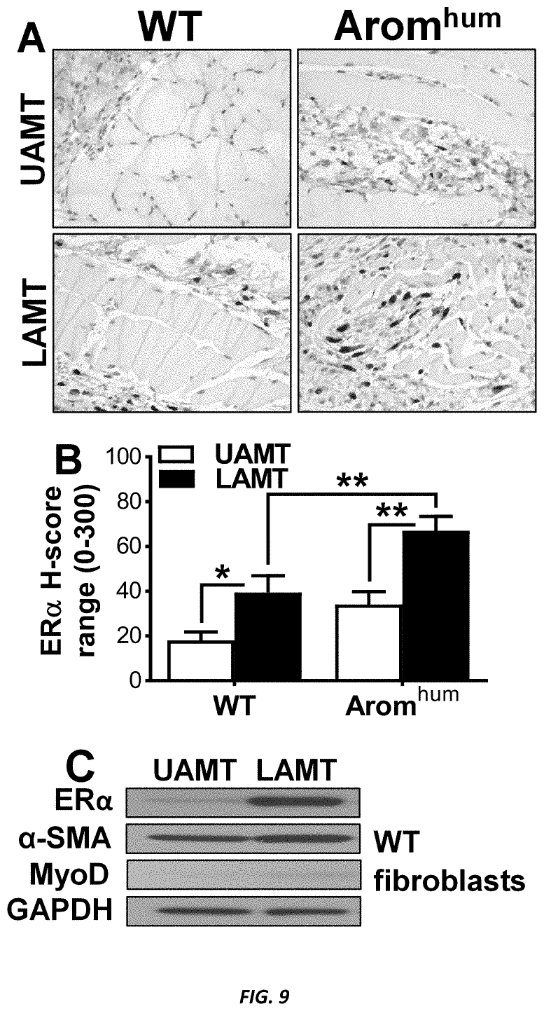

LAMT is extremely sensitive to estrogen due to extremely high ERα expression in its fibroblast component. qPCR showed that ERα is the only readily detectable estrogen receptor in some of the mouse and human skeletal muscle tissues, whereas ERα and Gpr30 were undetectable or barely detectable. We used IHC to localize ERα protein expression in various cell types in LAMT and UAMT. Nuclear immunoreactive ERα was primarily observed in fibroblasts dispersed in perimysial or endomysial stromal tissue but not in myocytes (FIG. 9A). The percentage of ERα-positive stromal cells in LAMT was significantly higher than that in UAMT in both WT and Arom^{hum} mice (FIG. 9B). ERα staining was negligible in fibroblasts of quadriceps muscle tissues (data not shown). In isolated fibroblasts from UAMT and LAMT of WT mice, ERα protein expression was significantly higher in LAMT fibroblasts (FIG. 9C). Together, these results suggest that higher fibroblastic ERα expression in LAMT is responsible for its higher sensitivity to locally produced E2 leading to fibrosis. Diffusely increased disorganized fibroblasts in LAMT are compounded by dispersed myocytes with small cytoplasms, termed myocyte atrophy, leading to hernia formation.

In summary, our preliminary studies provide novel evidence linking the aromatase expression and local E2 production in LAMT to muscle weakness and hernia development. We found that ERα is particularly enriched in LAMT fibroblasts of both male WT and Arom^{hum} mice, which makes it particularly sensitive to E2. Thus, we hypothesize that expression of human aromatase in LAMT of male Arom^{hum} mice converts T to E2, which induces ERα-rich fibroblast proliferation and ECM formation in LAMT, leading to replacement of myofibers by proliferating fibroblasts and ECM. Weakening and decreased tone of LAMT permits formation of inguinal hernias in all Arom^{hum} mice. The prevalence and timing of inguinal hernias correlate with the level of skeletal muscle aromatase expression and the advent of testicular secretion of T that is the substrate for aromatase (after 4 weeks of age). Conversely, no hernias develop in WT mice, which lack the human aromatase gene and thus do not express aromatase in muscle tissue. We further hypothesize that mice treated with an aromatase inhibitor or an E2/ERα antagonist will show diminished LAMT fibrosis and atrophy and a lower prevalence of inguinal hernia.

Experimental Design

Experiment 1a1: Does aromatase inhibition arrest or reverse LAMT fibrosis, atrophy, and hernia in male Arom^{hum} mice? None of the Arom^{hum} mice developed abdominal bulging before 3 weeks; all showed bulging by 8 weeks and readily visualized inguinal hernias by 12 weeks. Our preliminary data suggest that treatment with the aromatase inhibitor letrozole to inhibit estrogen production starting at 3 weeks of age (before hernia development) for 12 weeks prevents abdominal bulging, LAMT stromal fibrosis, myocyte atrophy, and inguinal hernias in three lines of Arom^{hum} mice studied (FIG. 5). However, we do not know the therapeutic effects of letrozole on the various stages of hernia development. The hernias will be classified as mild, early moderate, late moderate and severe if the bulging area through muscle tissue is ≤100 mm² (˜4 weeks), 100-150 mm² (˜6 weeks), 150-200 mm² (˜8 weeks), or ≥200 mm² (˜10 weeks), respectively. First, we will treat Arom^{hum} mice showing mild hernias with letrozole (SC 10 μg/day release pellet) or vehicle starting at 4 weeks of age for 12 weeks⁶³. We will assess whether muscle fibrosis, atrophy, and hernia can be arrested or reversed in mildly (<100 mm²) affected animals. If early letrozole treatment can arrest mild muscle fibrosis, we will delay the initiation of treatment to 6, 8, or 10 weeks of age to determine whether letrozole can arrest or reverse the growth of the early moderate (6 weeks), late moderate (8 weeks) or severe hernia (10 weeks). Hernia development will be monitored by visual inspection twice a week for 12 weeks after letrozole administration and will be reported as hernia incidence using Kaplan-Meier curve analysis. The length and width of the observed hernias will be measured to calculate the surface area (length×width). After completion of the treatment, the mice will be dissected, and LAMT, UAMT (control), and serum will be collected. One hundred mg of frozen muscle tissue will be obtained to measure tissue estrogen levels. Circulating and tissue (LAMT and UAMT) E2 levels will be measured at the IIT Research Institute by Dr. Miguel Muzzio (see letter) using LC-MS². We will use immunoblotting and IHC to determine ERα expression in LAMT and UAMT.

Does letrozole administration inhibit fibroblast proliferation, collagen formation, and fibrosis? To define the effect of letrozole administration on stromal cell proliferation, estrogen target gene expression indicating cell proliferation (e.g., Ccnd1, Greb1, and Pgr) will be measured in LAMT and UAMT homogenates using qPCR and in paraformaldehyde-fixed tissues using IHC. Additionally, proliferation of LAMT stromal cells will be measured by Ki67 IHC staining and PCNA immunoblotting. Next, we will use H&E staining to morphologically assess stromal fibrosis in muscle tissue. Masson's trichrome staining (MTS) will be performed to further determine the extent of ECM deposition in hernia tissue; and fibrotic areas (blue color) will be quantified using ImageJ software.

We will measure total amount of collagen (largely type I and III), a major component of ECM, through measuring the content of hydroxyproline⁶⁴. Type I collagen (Col1a1), the major collagen secreted by fibroblasts of fibrotic tissue, will be further determined by IHC and immunoblotting. We will also examine the expression of the newly identified skeletal muscle fibrosis-related genes (Kiss1, Ren1, Emb, Timp1, Spon2, Eln) in LAMT and UAMT after letrozole treatment using qPCR and IHC. These results will determine if decreased E2 by letrozole treatment reduces LAMT stromal cell proliferation, collagen excess, and fibrosis. Total collagen content will be measured by Dr. Lieber (co-PI), who is an expert in muscle ECM and cellular mechanisms that contribute to fibrosis¹⁴⁻¹⁹.

Does letrozole inhibit or reverse muscle atrophy and restore muscle strength? We will also perform MTS in LAMT and UAMT and histologically determine CSA of myofibers in muscle sections employing Axiovision 4.5 software (ZEISS) to assess muscle atrophy (FIG. 6). The myofiber marker, myosin heavy chain (Myh) will be measured in LAMT and UAMT using qPCR and immunoblotting. The muscle fibers we will assess are composed of transverse abdominis and the internal and external oblique muscles in healthy LAMT and UAMT as well as the atrophied remnants of these myofibers in estrogenized LAMT. We will quantify the LAMT architecture properties (e.g., physiologic CSA, fascicle length, and sarcomere length) to predict the maximum force-generating capacity of LAMT^{65, 66}. Muscle architecture analysis will be performed by Dr. Lieber (co-PI), who is an expert in muscle biomechanics^{65, 66}. Histologic assessment will be performed by Dr. E. McNally (consultant), who is an expert in muscle atrophy (Northwestern, see letters). The results will determine whether letrozole treatment inhibits LAMT muscle atrophy or reverses muscle strength.

Experiment 1α2: Does the general E2 antagonist fulvestrant, which downregulates and blocks ER, or ERα-selective E2 antagonist MPP prevent, arrest or reverse LAMT muscle fibrosis, atrophy and inguinal hernias in male Arom^{hum} mice? We found that ERα is the primary estrogen receptor in abdominal muscle fibroblasts and its levels are strikingly higher in LAMT vs. UAMT in Arom^{hum} mice (FIG. 9). Treatment of Arom^{hum} LAMT primary fibroblasts with fulvestrant (ICI 182,780), a general and strong antagonist that blocks ER-dependent E2 action via downregulation or inhibition of ER protein in target cells^{67, 68}, or MPP, an ERα-selective E2 antagonist⁶⁹⁻⁷¹, significantly decreased E2-induced expression of estrogen-responsive genes (Pgr and Greb1) and fibrotic genes (Spon2, Kiss1 and, Eln) (FIG. 10). Here, we will define whether the effects of E2 on hernia formation in LAMT is mediated via ERα in vivo. We will treat Arom^{hum} or WT mice with vehicle, fulvestrant (ICI 182,780), or MPP. The anti-estrogenic effects of fulvestrant will likely be mediated primarily via ERα in LAMT due to extremely high ERα:ERβ ratio and very low ERα levels. Furthermore, we will use an ERα-selective E2 antagonist to assess the roles of E2/ERα in hernia formation. First, we will administer fulvestrant (SC 1 mg/day release pellet)⁷² or MPP (SC 25 μg/day release pellet)^{73, 74} starting at 3 weeks of age for 12 weeks. Second, if fulvestrant or MPP treatment initiated at 3 weeks prevents muscle fibrosis, atrophy and hernias, we will start treatment at later time points (based on Experiment 1a1 results) to determine whether the treatment effect of fulvestrant or MPP on arresting or reversing the developing hernia is similar to that in letrozole treatment group. Third, sole treatment may not be able to arrest or reverse hernias. We will combine letrozole with fulvestrant or letrozole with MPP to determine whether the combined treatment exhibits the synergistic effects on the arrest or reversal of hernias at various stages. We will treat Arom^{hum} mice with vehicle, letrozole plus fulvestrant, or letrozole plus MPP at various stages for 12 weeks. We will also add selective ERβ antagonist PHTPP (subcutaneous 62.5 μg/day release pellet)⁷⁵ and high affinity and selective Gpr30 antagonist G-15 (subcutaneous 2.8 mg/day release pellet)⁷⁶ as (negative) controls since the expression of both ERβ and Gpr30 in LAMT is extremely low as compared to ERα. We will monitor hernia development, measure surface area of LAMT/hernia tissue, and histologically determine CSA of muscle fibers, analyze LAMT architecture properties (e.g., physiologic CSA, fascicle length, and sarcomere length) to assess LAMT fibrosis, atrophy, and strength. To assess the reversal of the hernia phenotype, Kaplan-Meier curve analysis will be used. Circulating and muscle tissue E2 levels will be measured using LC-MS². To validate the effects of each treatment, protein expression of ERα and E2/ERα target genes (Ccnd1, Pgr, and Greb1), and cell proliferation markers (Ki67 and PCNA) will be determined by IHC and immunoblotting. Fibrotic-related genes (Kiss1, Ren1, Emb, Timp1, Spon2, and Eln), the ECM marker Col1a1, and the myocyte marker Myh will be assessed using qPCR and IHC in LAMT after fulvestrant, MPP, and/or letrozole treatment. For statistical methods and power analyses, see Statistical Methods under Overall Strategy above.

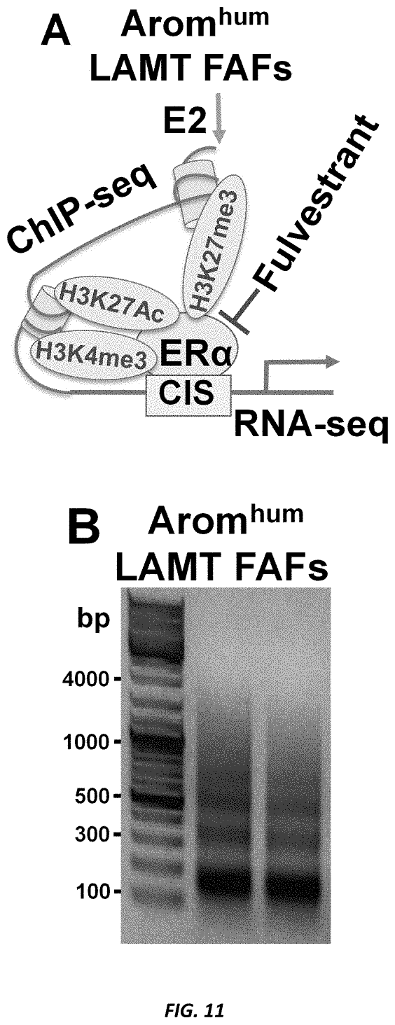

Hypothesis 1b. E2-activated ERα interacts with specific regions of chromatin and drives the expression of a distinct set of genes in LAMT fibroblasts from Arom^{hum} mice, leading to fibroblast proliferation, ECM production and hernia formation. We expect that some of those genes are potential targets for novel therapeutic options that can prevent recurrent inguinal hernia. Thus, we will determine the genome-wide ERα cistrome, histone modification maps, and transcriptomes in LAMT fibroblasts of Arom^{hum} mice (FIG. 11A).

Rationale. Evidence from ERα-positive breast cancer tissues reveals the critical roles of coordinated genome-wide ERα-chromatin interaction, histone modifications, and transcriptional activity in disease development⁷⁷. Modification of histones can establish patterns of gene expression. Histone H3 lysine 27 trimethylation (H3K27me3) is associated with repressed chromatin, while histone H3 lysine 4 trimethylation (H3K4me3) and histone H3 lysine 27 acetylation (H3K27ac) are associated with active promoters and enhancers, respectively⁷⁸⁻⁸². However, chromatin-based gene regulation by ERα in skeletal muscle fibroblasts is unknown, thereby hindering our understanding of the mechanisms underlying fibroblast proliferation, ECM production and hernia formation. Fibroblasts are usually quiescent, but hormonal stimuli may activate them. Persistent stimuli may epigenetically transform these activated fibroblasts to fibrosis-associated fibroblasts (FAFs) with enhanced proliferative properties⁸³. LAMT fibroblasts are quiescent in WT mice and become FAFs in Arom^{hum} mice under the influence of E2. Indeed, LAMT FAFs from Arom^{hum} express strikingly higher levels of ERα, grow faster, and produce more ECM compared to LAMT fibroblasts from WT mice (FIGS. 6-8). Although we are capable of performing whole-tissue ChIP-seq, we will define the genome-wide ERα-related molecular mechanisms involving LAMT fibrosis in primary LAMT fibroblasts (passage 0) because ERα in LAMT is almost exclusively expressed in fibroblasts (FIG. 9A). This approach will minimize the confounding effects of cellular heterogeneity between the WT LAMT composed largely of ERα-deficient myocytes and the fibroblast-rich Arom^{hum} LAMT. Thus, we will isolate LAMT FAFs from Arom^{hum} mice and incubate them with E2 or its antagonist fulvestrant to reveal the genome-wide underlying molecular mechanisms for fibrosis in Arom^{hum} mice.

Experiment 1b1: Using ChIP-seq, we will map global ERα binding sites on chromatin isolated from LAMT FAFs (passage 0) of 4-week old Arom^{hum} mice treated for 24 h with vehicle, E2 (10⁻⁸ M), or the potent E2 antagonist fulvestrant (10⁻⁷ M), that physically and functionally inhibits ERα (n=4 mice/group). Early herniation in Arom^{hum} mice usually become visible around 4 weeks of age (FIG. 5). Thus, we will isolate LAMT transformed FAFs at 4 weeks of age. We routinely culture primary fibroblast cells from LAMT of both WT and Arom^{hum} mice (FIGS. 6-9). The quantity of FAFs (>10⁷ cells/Arom^{hum} mouse) is sufficient for performing the proposed experiments. Previously, we successfully used two antibodies for ERα ChIP-seq or other genome-wide ChIP approaches^{84, 85}. We will perform optimization studies (ChIP-western and conventional ChIP) with both antibodies in primary muscle fibroblasts and use the most suitable antibody for ChIP-seqm. ChIP DNA will be prepared using the SimpleChIP Enzymatic Chromatin IP Kit (Cell Signaling, FIG. 11B); libraries will be prepared using Kapa Hyper Prep Kits (Kapa Biosystems). Library quality will be assessed using the Agilent 2100 Bioanalyzer. Sequencing will be performed at the Next-Generation Sequencing Core at Northwestern to obtain 30 million single-end 75-bp reads per sample. To add independent epigenetic criteria to assess the functionality of the identified ERα binding sites, we will perform ChIP-seq for H3K27me3, H3K4me3, and H3K27ac. By integrating histone modification signatures and global ERα-binding sites, we will define regulatory regions specifically activating or repressing gene transcription in LAMT FAFs of Arom^{hum} mice. Motif analysis will be used to discover novel transcription factors enriched in these unique regulatory regions. After integration with RNA-seq data and pathway analysis, functionally relevant and select histone modification or receptor binding sites will be confirmed using the traditional ChIP assay.

Experiment 1b2: We will perform RNA-seq at 40 million reads per sample on the 12 LAMT FAF samples treated for 24 h with vehicle, E2 (10⁻⁸ M), or fulvestrant (10⁻⁷ M) (n=4 mice/group). RNA will be isolated using the RNeasy Mini Kit (Qiagen) and libraries will be made using the Kapa Stranded RNA-seq with RiboErase kit. The library quality check and sequencing will be performed as described above (see Experiment 1b1). The RNA-seq data will be evaluated using pathway analysis and gene enrichment strategies with respect to the differentially expressed (DE) transcripts/genes induced by E2 and reversed by fulvestrant.

Bioinformatics analysis for ChIP-seq, RNA-seq, and their integration. Dr. Dai's lab at the University of Illinois at Chicago will perform the bioinformatics analysis (see letter). Drs. Dai and Bulun have had a long-standing collaboration and have coauthored papers in this area^{86, 87}. For ChIP-seq, raw reads will be first confirmed employing FastQC (http: //www.bioinformatics.babraham. ac. uk/projects/fastqc/). The deduplicated reads will be mapped to the Human Genome using Bowtie for the ChIP-seq reads, and peak calling will be performed using MACS⁸⁸. The differential binding (DB) sites will be detected using csaw⁸⁹. The motifs enriched adjacent to the binding sites will be analyzed using Homer (http://homer.salk.edu/homer/). The RNA-seq reads will be mapped and quantified using an ultra-fast tool Kallisto⁹⁰. The DE transcripts/genes will be identified using edgeR⁹¹. Both csaw and edgeR are designed for complex experimental approaches with replicates. We will use binding and expression target analysis (BETA) to integrate the DB sites and DE transcripts/genes⁹². This will prioritize ERα binding sites that directly regulate functional mRNA transcription and pathways that coordinate the concerted series of molecular events leading to muscle fibrotic process⁹³.

The integrated strategies are superior to singular approaches because they (1) distinguish active vs. repressive regulators, (2) identify novel transcription factor binding motifs as well as their co-factors, and (3) reduce false positive findings, thus enabling the design of effective validation experiments. Target genes will be validated using real-time PCR, immunoblotting, and IHC. Functional analysis of these genes will be performed in vitro (e.g., gene knockdown or overexpression) to determine their effects on proliferation and ECM formation.

Aim 1: Expected Results. We expect that ERα will be highly expressed in fibroblasts of LAMT compared to UAMT. ERα is anticipated to be negligible in myocytes. We anticipate that the aromatase inhibitor letrozole will prevent hernias if started as early as 3 weeks of age; and will arrest further enlargement of mild- and early moderate-size hernias developed before 6 weeks in Arom^{hum} mice (FIG. 5). The E2 antagonist fulvestrant and ERα antagonist MPP will also prevent or arrest muscle atrophy or hernia formation as letrozole does. We do not expect that letrozole, fulvestrant or MPP alone will reverse advanced inguinal hernias with protruding muscle areas >200 mm². We anticipate that combined treatment (letrozole plus fulvestrant or MPP) may be more effective than individual treatments for arresting advanced hernias. Letrozole will likely drastically decrease tissue E2 levels and mildly decrease circulating E2 in Arom^{hum} mice. The combined ChIP-seq and RNA-seq strategy will provide a functional understanding of how the nuclear receptor ERα□ in cooperation with histone modifications, determines the transcriptional activities of groups of genes that induce FAF proliferation and ECM production, leading to hernia formation. We expect to detect differentially active (H3K4me3 and H3K27ac) and repressive (H3K27me3) histone modification signatures in E2- and fulvestrant-treated FAFs, respectively. Particularly, in E2-treated FAFs, we anticipate the identification of a distinct set of ERα binding sites, which are modified by the active histone mark H3K4me3 and H3K27ac, leading to the transcription of proliferative and profibrotic genes. We expect that fulvestrant treatment will block or partially reverse E2-induced ERα binding and histone modification, and alter gene expression profiles (e.g., estrogen-responsive genes and fibrotic genes) in LAMT FAFs⁹⁴. The addition of motif analysis data will allow us to more accurately identify critical and novel transcription factors that are key for FAF activation, fibrosis and eventually hernia formation.

Aim 1: Potential Pitfalls and Alternative Strategies. Our team successfully used all proposed techniques including genome-wide ERα-chromatin interactions using ChIP-cloning or ChIP-seq strategies and bioinformatics analyses^{44, 45, 87, 95-98}. Considering cost and efficiency, we have selected 3 representative histone marks that provide limited assessment of active or repressed gene status; consequently, we will not detect all key epigenetic marks under different treatment conditions in muscle fibroblasts. In case the proposed bioinformatics analysis would not be suitable because of data distribution, we will use alternative approaches^{86,99-102}. Conceptually, we present compelling data to support the primary role of estrogen excess in muscle fibrosis and hernia development. In Arom^{hum} mice, serum T levels and androgen-responsive gene expression in LAMT are also lower compared with WT mice (data not shown). Studying the potentially contributory roles of low T and LAMT AR (located in myocytes) is beyond the scope of the current application and will be pursued as a future direction.

Aim 2. Determine whether conditional knockout of ERα in fibroblasts diminishes muscle fibrosis, myocyte atrophy or hernia and associate the mechanistic data from mouse experiments with human disease.

Hypothesis 2a. ERα, expressed in strikingly high levels in LAMT fibroblasts (FIG. 8), is responsible for mediating the effects of E2, causing fibroblast proliferation, ECM formation, myocyte atrophy, and hernia formation.

Rationale. E2, the biologically active estrogen, plays essential physiologic roles in both men and women^{31, 51} but also induces fibrosis in various pathologic tissues^{53, 54}. Expression of both ERα and ERβ has been reported in skeletal muscle^{103, 104}. We, however, found that mRNA levels of ERα in abdominal muscle tissues are >1000-fold and >200-fold higher than that of ERβ and Gpr30, which are absent or barely detectable, indicating that ERα is the major receptor type in this tissue. Estrogen sensitivity, i.e., ERα levels, in musculoskeletal tissue may increase with closer proximity to the pelvis, as estrogen during pregnancy has a general relaxing effect on pelvic muscles, ligaments, and bones, possibly in preparation for labor^{36, 37, 105} Although males do not go through pregnancy or labor, the evolutionary development of the tissue distribution of ERα may be similar in both sexes. Consistent with this, we found strikingly higher ERα levels in the LAMT stromal component compared with UAMT in both WT and Arom^{hum} male mice (FIG. 9). ERα levels are strikingly higher in LAMT fibrosis-associated fibroblasts (FAFs) of Arom^{hum} compared with the quiescent fibroblasts in WT LAMT (FIG. 9). ERα levels in quadriceps muscle, on the other hand, were negligible. Consequently, local formation of E2 in the LAMT of male Arom^{hum} mice may drive FAF proliferation, fibrosis, muscle atrophy, and hernia development via activation of ERα-rich LAMT FAFs (FIG. 9). ERα is expressed in the majority of the fibroblasts in LAMT (FIG. 9). Transient ERα knockdown significantly inhibits the expression of estrogen responsive- and fibrotic genes (FIG. 13). Moreover, fibroblast-specific protein 1 (Fsp1, also called S100A4) is co-localized with ERα in the majority of Fsp1-positive fibroblasts in LAMT (FIG. 11). This remarkable co-expression of ERα and Fsp1 will make it possible to test the role of ERα in hernia formation using a genetic approach employing Fsp1-Cre transgenic mice. Here, we will define the role of fibroblast-specific ERα for mediating the effects of aromatase and local E2 formation on LAMT fibrosis, myocyte atrophy and hernia formation. Prevention of fibrotic muscle atrophy and hernia via genetic disruption of ERα in Fsp1-positive fibroblasts will provide rigorous evidence for the role of estrogen action in muscle fibroblasts in hernia formation.

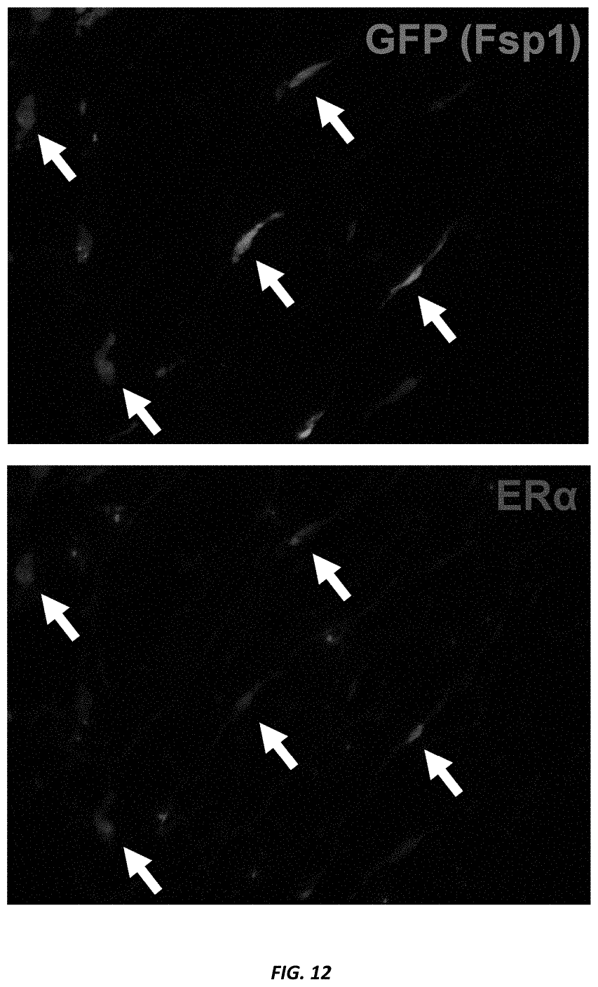

Preliminary Data: ERα co-localizes with Fsp1-positive fibroblasts, but not myocytes in LAMT. LAMT from 3-month-old Fsp1-GFP transgenic mice was analyzed for double immunofluorescence (IF)¹⁰⁶. Double IF staining of ERα with Fsp1 (GFP) in LAMT was performed to determine whether ERα is expressed in fibroblasts. We demonstrated nuclear ERα in the majority (>90%) of Fsp1-positive fibroblasts (FIGS. 9 and 12). ERα was undetectable in desmin-positive myocytes (control, data not shown).

In vitro transient ERα knockdown effectively inhibits the expression of estrogen-responsive and fibrotic genes. To define if ERα expression in LAMT fibroblasts is necessary for estrogenic effects, we transiently depleted ERα in Arom^{hum} LAMT fibroblasts using two mouse ERα (Esr1) siRNAs. Transient depletion of ERα significantly downregulated the expression of two estrogen-responsive genes (Pgr and Greb1) and two fibrotic genes (Kiss1 and Spon2) in the presence of E2 treatment (FIG. 13).

Experiment 2a: Does fibroblast-specific ablation of ERα prevent LAMT fibrosis and hernia in Arom^{hum} mice? We will obtain floxed ERα mice (ERα^{fl/fl}) from Dr. Radovick (see letter)¹⁰⁷ and Fsp1-Cre mice from the Jackson Laboratory (Jax #012641). We will cross these two mice to genetically ablate the ERα gene only in mouse fibroblast cells expressing Fsp1 fERα^{-/-}). In addition to confirmation with genotyping, we will also validate efficiency of recombination (ablation) between two loxP sites in fibroblasts using Fsp1/ERα double IF, which will be essential for interpreting the results. Once we confirm generation of the fERα1^{-/-} mouse, we will cross it with the Arom^{hum} mouse to generate a mouse lacking fibroblast ERα in LAMT (and possibly other tissues) and expressing a single human aromatase allele (fERα^{-/-}-Arom^{hum}). fERα^{+/+}-Arom^{hum} and fERα^{-/-}-Arom^{hum} littermates will be used as controls.

Do fERα^{-/-}-Arom^{hum} mice exhibit decreased E2/ERα action in muscle fibroblasts and not in other cell types? ERα mRNA and protein will be assessed in LAMT homogenates by qPCR and immunoblotting. Since LAMT homogenates are mixtures of fibroblasts and myocytes, the extent of ERα deletion in fibroblasts may not be shown precisely. LAMT will be paraffin-embedded and sectioned for IHC localization of ERα to confirm deletion in fibroblasts. To define if ERα is fully deleted in fibroblasts, primary LAMT fibroblasts (passage 0) will be isolated and cultured from the LAMT of fERα^{-/-}-Arom^{hum} mice and controls, and ERα mRNA and protein levels will be measured using qPCR and immunoblotting. We have successfully cultured primary fibroblasts from mouse tissues including LAMT (see FIG. 9)^{44, 108, 109}. In brief, LAMT will be minced and digested with collagenase D (1.5 U/ml), dispase II (2.4 U/ml), and CaCl₂ (2.5 mM) at 37° C. for 30 minutes. Single-cell suspensions will be prepared by filtration through a 75-μm sieve.

Fibroblasts in cell suspensions will be allowed to attach to collagen-coated petri dishes for 15 min and unattached myoblasts will be removed. Primary fibroblasts will be obtained by growing cells in DMEM/F-12 with 10% FBS, which preferentially supports growth of fibroblasts. To confirm that the fibroblast-specific deletion of ERα decreases E2/ERα action, E2-responsive mRNA/protein expression (e.g., Ccnd1, Greb1, and Pgr) will be determined in LAMT or UAMT fibroblasts after E2 (10⁻⁸ M) treatment for 24 h. We will measure serum and muscle tissue E2 levels by LC-MS².

Does fibroblast-specific ablation of ERα decrease fibroblast proliferation and profibrotic gene expression? LAMT fibroblast proliferation will be measured by Ki67 IHC staining and PCNA immunoblotting of tissues from fERα^{-/-}-Arom^{hum} mice or their littermate controls. mRNA and protein levels of fibrosis-related genes (e.g., Kiss1, Ren1, Emb, Timp1, Spon2, Eln, and Col1a1) will be assessed in UAMT/LAMT by qPCR and IHC and in E2-treated primary fibroblasts from LAMT using immunoblotting.

Does fibroblast-specific ablation of ERα diminish LAMT fibrosis, atrophy, and hernia formation? Hernia formation will be monitored twice a week by visual inspection from 3 to 26 weeks of age. Hernia incidence, time to hernia onset, and hernia surface area will be analyzed by Kaplan-Meier plot. At euthanasia, muscle-fiber CSA and stromal ECM will be measured in Masson's trichrome stained-sections, and Col1a1 will be determined by IHC and immunoblotting to confirm the results from MTS. Total collagen content in LAMT will be defined by hydroxyproline assay and LAMT architecture properties will be analyzed, as described under Experiment 1a1. For statistical methods and power analyses, see Statistical Methods under Overall Strategy above.

Hypothesis 2b. The histologic and molecular changes in LAMT of Arom^{hum} male mice are present in tissues of a subset of elderly men with inguinal hernia.

Rationale. After exploration of causal mechanisms in mice, we will test whether these histologic and molecular changes are present in a subset of men with hernias. Conversion of circulating T to E2 via aromatase expression in bulky tissues, namely, skeletal muscle and adipose tissue, produces the majority of estrogen in men^{55, 56} Muscle and adipose tissue aromatase expression increases with advancing age; the rate of aromatization of T in these peripheral tissues in a 60+-year-old man (peak age for inguinal hernia) is approximately 4-fold higher than that in a 20-year-old man⁵⁷⁻⁵⁹. Notably, hernias in Arom^{hum} mice are first observed at 4 weeks, immediately after the start of secretion of testicular T, which is the substrate for muscle aromatase. The contrast between WT male mice with practically no peripheral aromatase expression or hernia development and Arom^{hum} mice with peripheral aromatase expression and a strikingly high occurrence of inguinal hernias is thus analogous to the contrast between young men with low peripheral aromatase expression and low incidence of hernia and a subset of older men with high peripheral aromatase expression and peak incidence of indirect inguinal hernias. Thus, we will examine the molecular signatures of human tissues and compare them to those of Arom^{hum} mice.

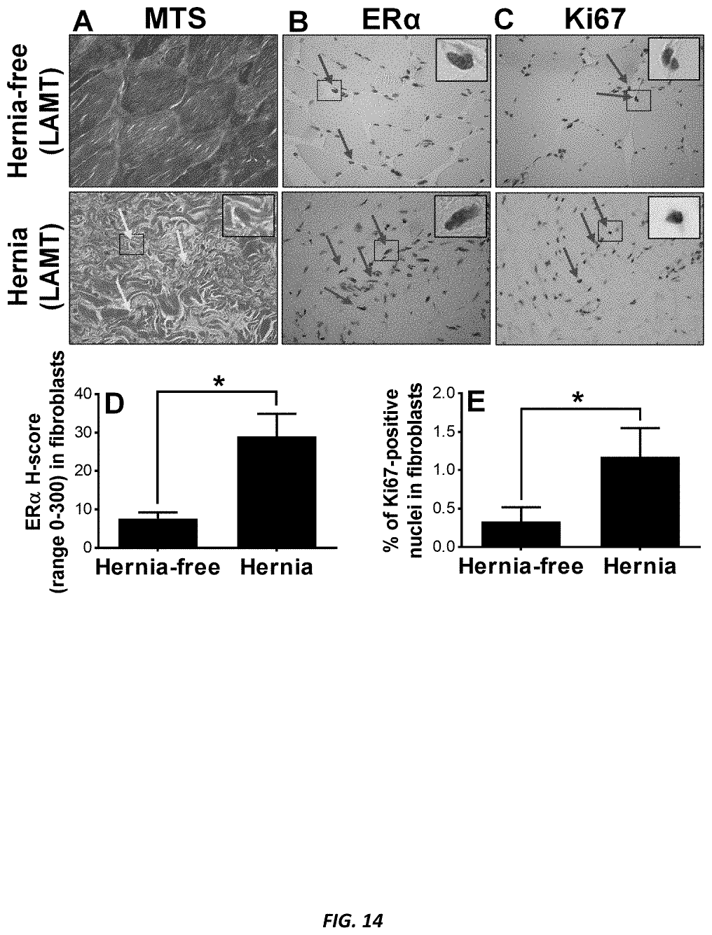

Preliminary Data. ERα expression and proliferative activity in normal human LAMT and LAMT affected by hernia. IHC for ERα was performed on LAMTs from men undergoing abdominal surgery for another benign indication and on fibrotic LAMTs from men undergoing hernia repair surgery. A total of 12 men were included in this study, 6 were hernia-free (control; 50-68 yr-old) and 6 had hernia (60-77 yr-old). Normal LAMTs consisted of healthy-appearing muscle fibers and stromal tissue, whereas LAMT removed as hernia wall consisted of fibrotic tissue with islands of atrophic myocytes (yellow arrows; FIG. 14A). Consistent with the mouse ERα expression pattern, the ERα H-score was strikingly higher in the stromal component of LAMTs from hernia patients compared with control men (FIG. 14). The stromal cell proliferation marker Ki67 immunoreactivity was significantly higher in hernia-wall LAMTs (1.2%) compared with normal LAMTs (0.3%; FIG. 14). These findings suggest a link between enhanced estrogen action and LAMT fibrosis associated with inguinal hernia in men.

Experiment 2b: Are the levels of aromatase, E2, and ERα in human LAMT/UAMT consistent with those observed in the mouse model of hernia? To define whether the mouse abdominal muscle tissue levels of aromatase, E2, and ERα mirror their expression/production pattern in men, our co-investigator Dr. Stulberg from General Surgery will obtain biopsies of LAMTs associated with hernia walls from men undergoing surgery for inguinal hernia or LAMTs/UAMTs of age-matched (40 to 80+ years old) hernia-free men undergoing abdominal surgery for another benign indication (The risks of obtaining UAMT samples from men undergoing hernia surgery do not justify the research benefits.) Men were required to have intact and healthy abdominal muscle tissue. Those excluded were men with a personal history of muscular dystrophy and atrophy, and recent abdominal surgeries, and those with any cancer types within the previous five years. Prior and current medications will be restricted as follows: hormone replacement therapy or topical hormonal preparation was not permitted within 12 months of study enrollment. Men will also be excluded if they had any condition that in the opinion of the investigator may not make it safe to take part. We will evaluate mRNA (qPCR) and protein levels (immunoblotting, IHC) of aromatase, ERα, and estrogen-responsive (CCND1, GREB1, and PGR) and fibrosis-related genes (KISS1, REN1, EMB1, TMP1, SPON2, ELN, and COL1A1) in the human tissue samples. We anticipate to augment the spectrum and pathologic relevance of ERα-target and profibrotic gene signatures based on the results from the ChIP-seq and RNA-seq experiments described under Experiments 1b1/1b2. E2 and T levels in both tissue and serum will be measured by LC-MS². A 36% increase in ERα in LAMT compared with UAMT can be reliably detected with approximately 4 men per group, assuming a coefficient of variation of 50% and a two-tailed, α:0.05-level, and independent-sample t-test. We plan to recruit a total of 30 men to each group (30 men with hernia and 30 hernia-free men) to ensure a realistic chance of obtaining meaningful results. We will use ANOVA followed by Tukey analysis for comparing the mRNA and protein levels of these genes in three groups of tissues: UAMT or LAMT from hernia-free patients and LAMT from hernia patients. We will also use Pearson's correlation analysis to determine whether tissue E2 levels, and aromatase/ERα expression and ERα-target and fibrosis-related gene signatures vary with age.

Aim 2: Expected Results. We anticipate that fibroblast-specific ablation of ERα in Arom^{hum} mice will prevent or delay LAMT fibrosis, muscle atrophy, and hernia formation. Because E2 acts via ERα in the fibroblast component of LAMT, ERα ablation will have a nearly complete protective effect against hernia formation. We expect that LAMT FAFs in fERα^{-/-}-Arom^{hum} mice will become less fibrotically active, i.e., decreased proliferation and ECM production. Fibroblast-selective ERα disruption will also restore the physiologic properties and strength of LAMT to normal levels. In a subset of elderly men with hernia, we expect slightly increased serum E2 (and lower T) as compared to younger controls with hernia; the magnitude of age-related increase in tissue (LAMT/UAMT) levels of E2 is expected to be much larger compared with its circulating levels due to strikingly elevated tissue aromatase. In this elderly group, we expect that the tissue E2 levels and expression patterns of aromatase, ERα and their target genes in LAMT/UAMT will be similar to those observed in Arom^{hum} mice. We expect that LAMT/UAMT levels of E2 and aromatase/ERα expression will increase with advancing age in a subset of men with hernia much more steeply compared with hernia-free men. Thus, E2/aromatase/ERα levels will be higher in LAMTs of hernia patients compared with age-matched hernia-free patients. ERα levels will be higher in LAMT compared with UAMT. E2/ERα-target and fibrosis-related gene signatures will follow these patterns.

Aim 2: Potential Pitfalls and Alternative Strategies. We have more than 15 years of experience in the described techniques^{44, 45, 50, 98, 108-111}. If ablation of fibroblast-specific ERα is incomplete or inefficient, we will use germline null mice to generate flox/-mice, making somatic mutation more efficient. In addition, we will mate the Cre-driver lines to homozygosity, raising the expression level of Cre-recombinase. The third alternative strategy will be used in case Fsp1-Cre does not effectively ablate ERα in LAMT fibroblasts. We will use Esr1-Cre (Jax #017911) to nonselectively ablate ERα in all ERα-expressing cells. Total ERα knockout may produce unintended consequences such as increased gonadotropins and testicular E2 secretion; despite these phenotypic alterations, however, total ERα ablation is expected to prevent hernia development in Arom^{hum} mice. We will increase the sample size or widen age-range if human studies show a large subject-to-subject variation.

Overall Impact and Future Directions. The etiology of inguinal hernia is unknown. After surgical repair, complications such as long-term postoperative pain, infection, and a high rate of recurrence continue to challenge the surgeons and their patients. Our studies will reveal the underlying molecular mechanisms for hernia development in men and provide the basis for developing novel pharmacological approaches for preventing recurrence of inguinal hernia at high risk individuals.

Our data clearly indicate that aromatase, E2 and ERα collectively play the central role in LAMT fibrosis and hernia phenotype. It will, however, be important to define the contributory role of androgen action in this paradigm. We found that AR is selectively expressed in myocytes of LAMT or other skeletal muscle groups and that serum T levels are somewhat lower in both Arom^{hum} mice and elderly men. Moreover, the androgen-responsive gene signature is significantly decreased in LAMT of Arom^{hum} mice (data not shown). In the future, we will define the effects of T or, the nonaromatizable androgen, DHT on LAMT using male mice of various genetic backgrounds. We will selectively delete AR in myocytes to dissect the roles of T or AR in hernia development. We will define the relative roles of ERα in fibroblasts and AR in myocytes in maintaining muscle (LAMT) function and in pathologic processes such as fibrosis, atrophy and hernia formation. We will extend our research to women to define the functions of E2/ERα in pelvic floor muscles.

### Example 2—Roles of Sex Steroid Hormones and their Analogues in the Prevention and Treatment of Hernias

Inguinal hernia is among the most diagnosed diseases in general surgery, and its repair is one of the most common operations in the world. Until recently, its etiology remains unknown. We demonstrated for the first time that the conversion of testosterone to estradiol (E2) by the aromatase enzyme in lower abdominal muscle tissue (LAMT) causes intense fibrosis, leading to muscle atrophy and inguinal hernia; an aromatase inhibitor entirely prevents this phenotype. This fibrotic mechanism is mediated by estrogen receptor α (ERα) located in the fibroblast component of the skeletal muscle tissue and is activated by estrogen produced from testosterone via local aromatization. These mice also have decreased circulating testosterone and increased muscle tissue estrogen levels, a hormonal profile that resonates with a subset of elderly men who are prone to hernia development. These findings have been published in Proceedings of the National Academy of Sciences of the United States of America (PNAS, 2018 Oct. 30; 115(44):E10427-E10436. doi: 10.1073/pnas.1807765115. Epub 2018 Oct. 16). Our continuing study demonstrates that modulation of estrogen action can not only prevent hernia formation but also reverse mild to large (severe) hernias.

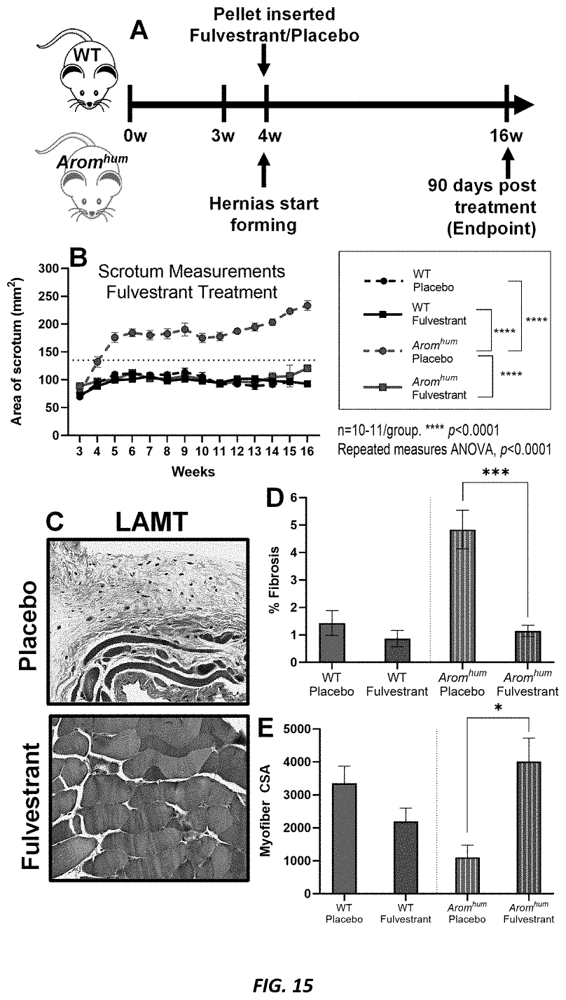

We previously developed a mouse model that expresses the human aromatase gene (Arom^{hum}) wherein all male mice develop inguinal hernias; aromatase inhibitor, letrozole, completely prevented the formation of inguinal hernias in Arom^{hum} mice. This finding was published in PNAS in October, 2018. Here we further show that similar to letrozole preventing hernia formation, ER-dependent E2 antagonist fulvestrant can also prevent LAMT fibrosis, muscle atrophy, and hernia formation in Arom^{hum} mice (FIG. 15). We treated Arom^{hum} mice with fulvestrant for 12 weeks starting at the age of 4 weeks (before hernia formation). As expected, placebo-treated Arom^{hum} mice developed scrotal hernias. However, the hernia formation was completely prevented by fulvestrant administration. Wild-type (WT) littermates did not show hernia formation with or without fulvestrant treatment (FIG. 15B). Development of LAMT fibrosis in Arom^{hum} mice was completely prevented with letrozole treatment (FIGS. 15C and D). The myofiber cross-sectional area (CSA) in untreated Arom^{hum} mice was significantly smaller than in WT mice, indicating muscle atrophy. Fulvestrant treatment completely prevented myofiber atrophy in Arom^{hum} mice (FIGS. 15C and E).

### Example 3—Estrogen Signaling Modulation is a Prospective Treatment for Inguinal Hernias

Background: Inguinal hernias are a widespread public health issue and typically diagnosed in one-fourth of all men. Despite hernia repair being the most commonly performed surgery in the US, the mechanisms causing this disease are currently unknown. We previously developed a mouse model that expresses the human aromatase gene (Arom^{hum}) wherein all male mice develop inguinal hernias. We further showed that high production of estradiol (E2) by aromatase in LAMT via binding to estrogen receptor (ER) caused increased fibroblast proliferation and muscle atrophy which leads to inguinal hernia formation.

Hypothesis: Disruption of estrogen signaling via ablation of estrogen production using an aromatase inhibitor or inhibition of estrogen receptor by an estradiol antagonist can prevent hernia formation or reverse the established inguinal hernias.

Results: We test three types of treatments to inhibit E2-ER signaling: letrozole, fulvestrant, and raloxifene. We previously demonstrated that aromatase inhibitor, letrozole, completely prevented the formation of inguinal hernias in Arom^{hum} mice (PNAS, 2018 Oct. 30; 115(44):E10427-E10436. doi: 10.1073/pnas.1807765115. Epub 2018 Oct. 16). We also show that ER-dependent E2 antagonist fulvestrant can also prevent LAMT fibrosis, muscle atrophy, and hernia formation in Arom^{hum} mice (FIG. 15). WT littermates did not show hernia formation with or without letrozole/fulvestrant treatment.

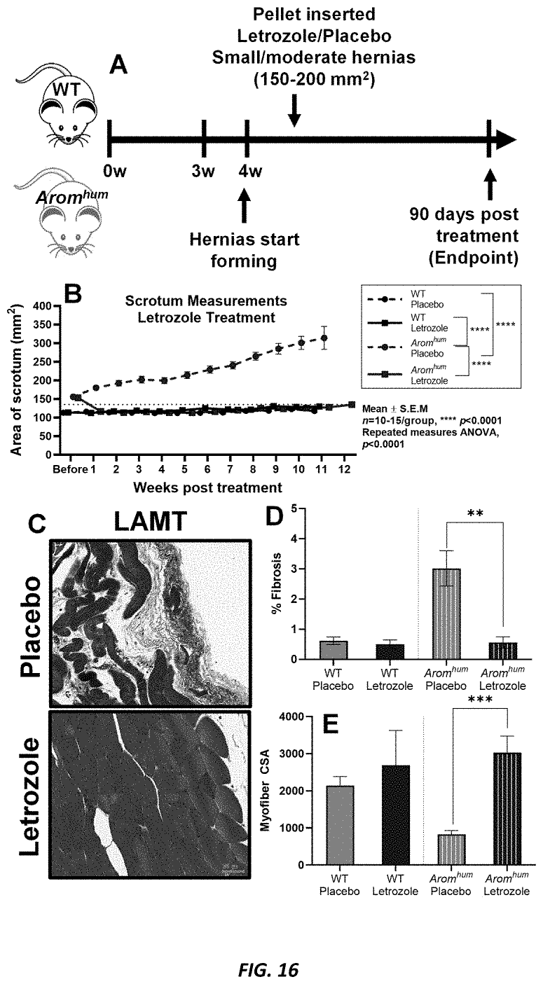

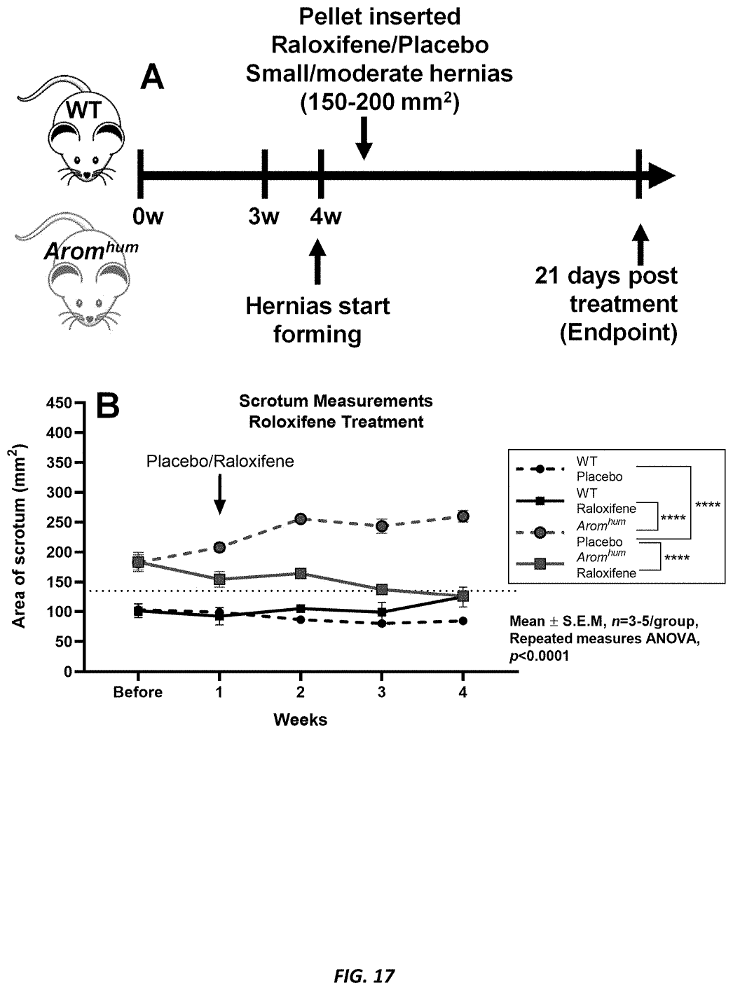

Furthermore, we demonstrate that aromatase inhibitor letrozole can reverse mild to moderate size of hernia (150-200 mm²), while placebo-treated mice had progressively enlarged hernias (FIG. 16). We subsequently show a reduction in muscle fibrosis and a restoration of myocyte size in Arom^{hum} mice with letrozole treatment. We treated Arom^{hum} mice with letrozole for 12 weeks. In addition, we treated Arom^{hum} mice with another E2-ER antagonist raloxifene for 21 days and demonstrate that raloxifene can also reverse mild to moderate size of hernia (FIG. 17). Raloxifene is a selective estrogen receptor modulator (SERM) and is currently used to prevent and treat osteoporosis in postmenopausal women.

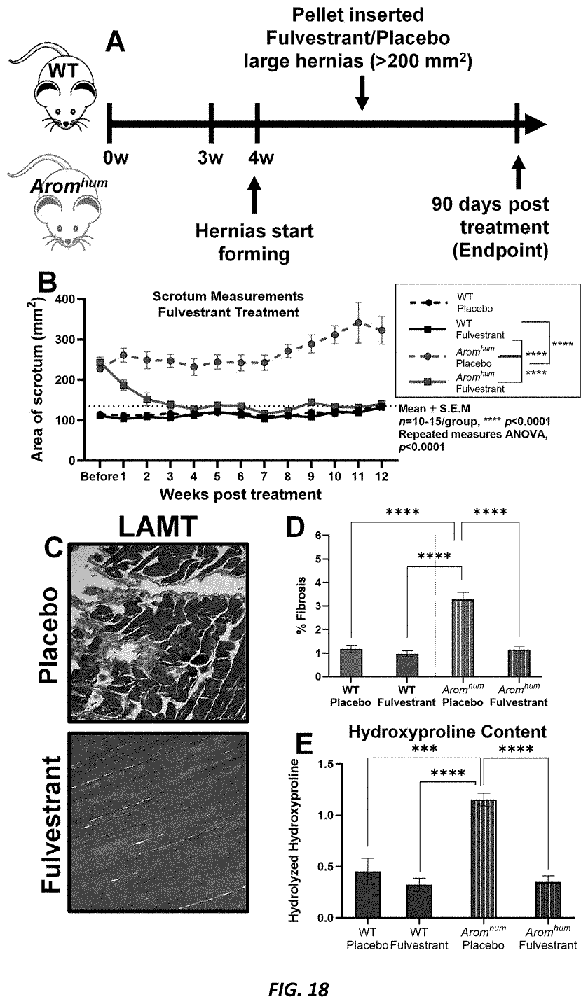

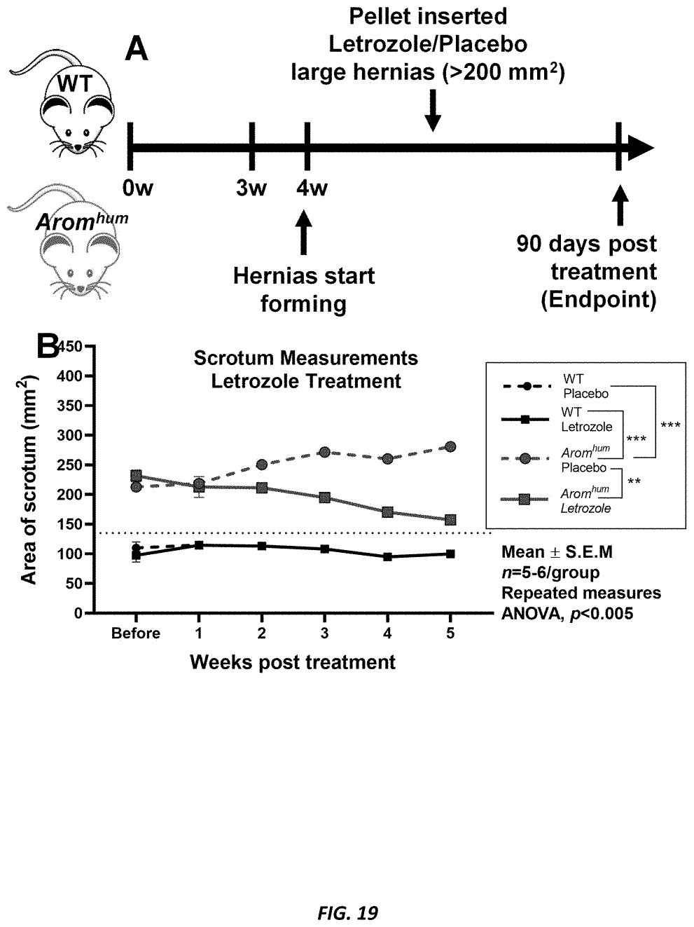

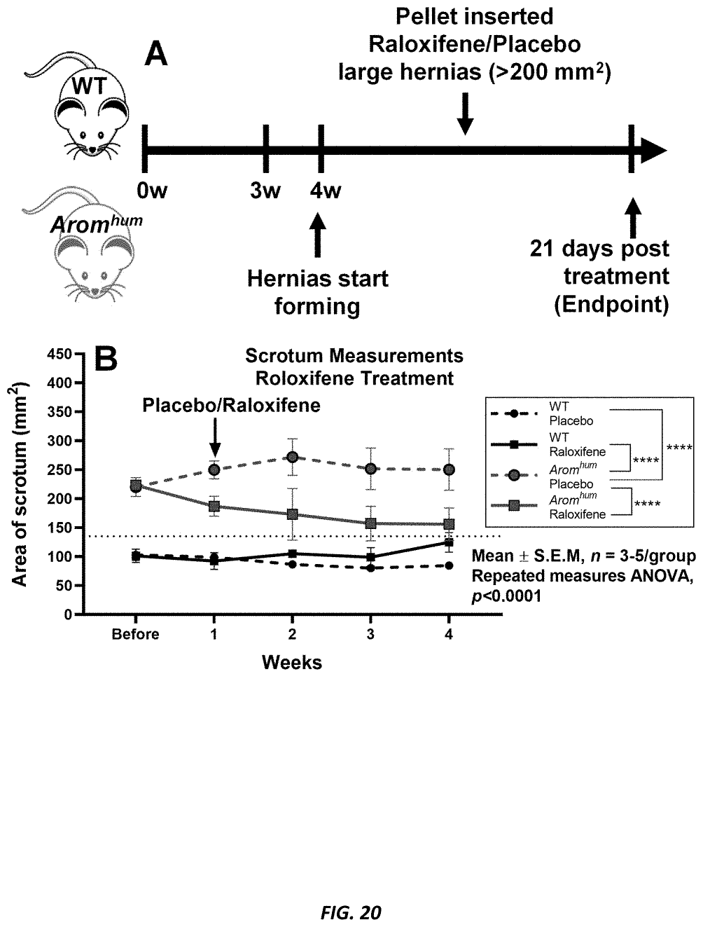

Most interestingly, sole fulvestrant treatment can reverse large and severe hernias (>200 mm²), accompanied by a decrease in muscle fibrosis and an increase in myofiber cross-section area compared to placebo mice (FIG. 18). We treated Arom^{hum} mice with fulvestrant for 12 weeks when the hernia area was over 200 mm². Large hernias were completely disappeared after 3- to 4-week of fulvestrant administration. WT littermates did not show hernia formation with or without fulvestrant treatment (FIG. 18B). Established fibrosis in the LAMT of Arom^{hum} mice was completely reversed with fulvestrant treatment (FIGS. 18C and D). We measured the total amount of collagen (largely type I and III), a major component of ECM, through measuring the content of hydroxyproline(1). Total collagen content in LAMT defined by hydroxyproline assay was higher in placebo-treated Arom^{hum} mice and decreased to normal levels similar to WT mice after fulvestrant treatment (FIG. 18E). Fulvestrant treatment completely reversed myofiber atrophy in Arom^{hum} mice (FIG. 18C). Similarly, Treatment with letrozole or raloxifene also reverses large scrotal hernia in Arom^{hum} mice (FIGS. 19 and 20). However, letrozole and raloxifene require a longer time to reverse large hernias as compared to fulvestrant treatment. 3-week raloxifene treatment was capable to reverse small to large hernias (FIGS. 17 and 20).

Conclusion: Estrogen produced as a result of aromatase expression in estrogen-sensitive LAMT stimulates the proliferation of estrogen receptor-expressing fibroblasts, fibrosis, muscle atrophy, and hernia formation. Ablation of estrogen production or its signaling not only completely prevents this phenotype but also reverses mild to large-sized hernias. Our findings pave the pathway for developing the first potential preventive and therapeutic pharmacological approach for combating recurrent inguinal hernias in elderly men through modulation of estrogen signaling in abdominal muscle tissue.

### Example 4—Defining the Role of Androgens in Hernia-Associated Skeletal Muscle Fibrosis and Hernia Development

Introduction: Inguinal hernia is a highly prevalent condition occurring in 27% of adult men in their lifetime. The recurrence rate of hernia is 5-20%, resulting in a substantial cost burden in surgical repair procedures. Until recently, the mechanisms leading to the lower abdominal muscle tissue (LAMT) weakening characteristic of hernia were unknown. Our group developed the first mouse model of inguinal hernia through expression of the human aromatase enzyme in male mice (Arom^{Hum}). Aromatase converts androgens to estrogens, and is expressed in the skeletal muscle tissue in humans, but not mice. We found that locally formed estrogen from aromatase activity in LAMT and decreased circulating testosterone levels in Arom^{Hum} mice are associated with muscle atrophy and fibrosis resulting in hernia. However, it is unclear how decreasing androgen levels might affect muscle fibrosis, and defining this potential mechanism could impact hernia treatment. We hypothesized that low androgen levels promote muscle fibroblast proliferation and fibrosis, and that androgen treatment would prevent hernia progression in Arom^{Hum} mice.

Methods: Arom^{Hum} mice (3 weeks old) were treated with high-dose dihydrotestosterone (DHT) via injection for 53 days with hernia volume continuously recorded (n=5/group). Primary fibroblasts were isolated from LAMT from WT and Arom^{Hum} mice (n=5/genotype). Cells were treated for 24 hours with increasing doses (0.001, 0.01, 0.1, 1, 5, 10, and 100 nM) of R1881, a synthetic androgen, and compared to untreated cells by western blot.

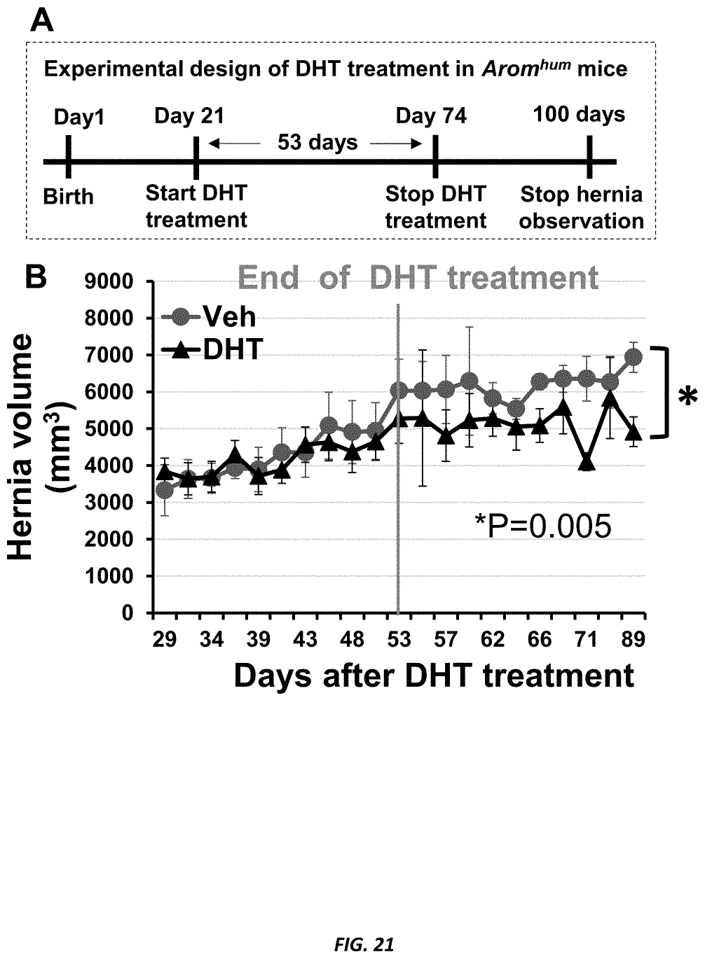

Results: Hernia volume was significantly decreased in Arom^{Hum} mice treated with DHT compared to vehicle-treated mice, and volume remained consistently suppressed after DHT treatment (p<0.005, FIG. 21). In both primary fibroblast lines, R1881 treatment increased AR levels in a dose-dependent manner, indicating that the treatment was effective. Preliminary data indicated that low doses of R1881 (0.001 and 0.01 nM) increased PCNA levels in LAMT WT and LAMT Arom^{Hum} fibroblasts. Densitometry normalized to GAPDH showed 80% and 60% increases for 0.001 nM and 0.01 nM respectively in LAMT WT fibroblasts, and 20% and 30% increases at these doses in LAMT Arom^{Hum} fibroblasts. Higher doses of R1881 decreased PCNA levels in LAMT Arom^{Hum} fibroblasts by 40% (10 nM) and 30% (100 nM), whereas a 25% decrease was detected in LAMT WT fibroblasts at 100 nM.

Conclusion: These data suggest that low androgen doses increase LAMT fibroblast proliferation, which possibly contributes to hernia formation. Androgen treatment at higher doses can partially block the progression of hernia in vivo. However, it is unclear whether and how androgen deficiency in combination with excess estrogen affects fibroblast proliferation and hernia formation. Additional research is required to further determine if androgen supplementation in sufficient doses is a potential therapeutic for inguinal hernia and other muscle weakness diseases.

### REFERENCES

1. Bay-Nielsen M, Kehlet H, Strand L, Malmstrom J, Andersen F H, Wara P, Juul P, Callesen T, Danish Hernia Database C. Quality assessment of 26,304 herniorrhaphies in Denmark: a prospective nationwide study. Lancet. 2001; 358(9288):1124-8. doi: 10.1016/S0140-6736(01)06251-1. PubMed PMID: 11597665.
2. Jenkins J T, O'Dwyer P J. Inguinal hernias. BMJ. 2008; 336(7638):269-72. doi: 10.1136/bmj.39450.428275.AD. PubMed PMID: 18244999; PubMed Central PMCID: PMC2223000.
3. Primatesta P, Goldacre M J. Inguinal hernia repair: incidence of elective and emergency surgery, readmission and mortality. International journal of epidemiology. 1996; 25(4):835-9. PubMed PMID: 8921464.
4. Kingsnorth A, LeBlanc K. Hernias: inguinal and incisional. Lancet. 2003; 362(9395):1561-71. doi: 10.1016/S0140-6736(03)14746-0. PubMed PMID: 14615114.
5. Aasbo V, Thuen A, Raeder J. Improved long-lasting postoperative analgesia, recovery function and patient satisfaction after inguinal hernia repair with inguinal field block compared with general anesthesia. Acta anaesthesiologica Scandinavica. 2002; 46(6):674-8. PubMed PMID: 12059890.
6. Matthews R D, Neumayer L. Inguinal hernia in the 21st century: an evidence-based review. Curr Probl Surg. 2008; 45(4):261-312. doi: 10.1067/j.cpsurg.2008.01.002. PubMed PMID: 18358264.
7. Malangoni M R, M. The Biological Basis of Modern Surgical Practice. In: Townsend C J B, D.; Evers, M.; Mattox, K.; eds., editor. Sabiston Textbook of Surgery. 18th ed. Philadelphia: Saunders Elsevier; 2008.
8. Matthews R D, Anthony T, Kim L T, Wang J, Fitzgibbons R J, Jr., Giobbie-Hurder A, Reda D J, Itani K M, Neumayer L A, Veterans Affairs Cooperative 456 Studies Program I. Factors associated with postoperative complications and hernia recurrence for patients undergoing inguinal hernia repair: a report from the VA Cooperative Hernia Study Group. Am J Surg. 2007; 194(5):611-7. doi: 10.1016/j.amjsurg.2007.07.018. PubMed PMID: 17936422.
9. Aasvang E, Kehlet H. Chronic postoperative pain: the case of inguinal herniorrhaphy. British journal of anaesthesia. 2005; 95(1):69-76. doi: 10.1093/bja/aei019. PubMed PMID: 15531621.
10. Yang B, Jiang Z P, Li Y R, Zong Z, Chen S. Long-term outcome for open preperitoneal mesh repair of recurrent inguinal hernia. International journal of surgery. 2015; 19:134-6. doi: 10.1016/j.ijsu.2015.05.029. PubMed PMID: 26021274.
11. Hellspong G, Gunnarsson U, Dahlstrand U, Sandblom G. Diabetes as a risk factor in patients undergoing groin hernia surgery. Langenbecks Arch Surg. 2017; 402(2):219-25. doi: 10.1007/s00423-016-1519-8. PubMed PMID: 27928640.
12. Belghiti J, Durand F. Abdominal wall hernias in the setting of cirrhosis. Semin Liver Dis. 1997; 17β):219-26. doi: 10.1055/s-2007-1007199. PubMed PMID: 9308126.
13. Andraus W, Pinheiro R S, Lai Q, Haddad L B P, Nacif L S, D'Albuquerque L A C, Lerut J. Abdominal wall hernia in cirrhotic patients: emergency surgery results in higher morbidity and mortality. BMC Surg. 2015; 15:65. doi: 10.1186/s12893-015-0052-y. PubMed PMID: 25990110; PubMed Central PMCID: PMC4443633.
14. Lieber R L, Ward S R. Cellular mechanisms of tissue fibrosis. 4. Structural and functional consequences of skeletal muscle fibrosis. Am J Physiol Cell Physiol. 2013; 305β):C241-52. doi: 10.1152/ajpce11.00173.2013. PubMed PMID: 23761627; PubMed Central PMCID: PMC3742845.
15. Gillies A R, Bushong E A, Deerinck T J, Ellisman M E I, Lieber R L. Three-dimensional reconstruction of skeletal muscle extracellular matrix ultrastructure. Microsc Microanal. 2014; 20(6):1835-40. doi: 10.1017/S1431927614013300. PubMed PMID: 25275291; PubMed Central PMCID: PMC4267978.
16. Gillies A R, Chapman M A, Bushong E A, Deerinck T J, Ellisman M H, Lieber R L. High resolution three-dimensional reconstruction of fibrotic skeletal muscle extracellular matrix. J Physiol. 2017; 595(4):1159-71. doi: 10.1113/JP273376. PubMed PMID: 27859324; PubMed Central PMCID: PMC5309386.
17. Gillies A R, Lieber R L. Structure and function of the skeletal muscle extracellular matrix. Muscle Nerve. 2011; 44β):318-31. doi: 10.1002/mus.22094. PubMed PMID: 21949456; PubMed Central PMCID: PMC3177172.
18. Chapman M A, Meza R, Lieber R L. Skeletal muscle fibroblasts in health and disease. Differentiation. 2016; 92β):108-15. doi: 10.1016/j.diff.2016.05.007. PubMed PMID: 27282924; PubMed Central PMCID: PMC5079803.
19. Chapman M A, Mukund K, Subramaniam S, Brenner D, Lieber R L. Three distinct cell populations express extracellular matrix proteins and increase in number during skeletal muscle fibrosis. Am J Physiol Cell Physiol. 2017; 312(2):C131-C43. doi: 10.1152/ajpce11.00226.2016. PubMed PMID: 27881411; PubMed Central PMCID: PMC5336596.
20. Abdeen N S, W. M. Review of Inguinal Region Hernias on MDCT. Applied Radiology. 2011; 40(4):6.
21. Youssef T, El-Alfy K, Farid M. Randomized clinical trial of Desarda versus Lichtenstein repair for treatment of primary inguinal hernia. International journal of surgery. 2015; 20:28-34. doi: 10.1016/j.ijsu.2015.05.055. PubMed PMID: 26074293.
22. Zendejas B, Ramirez T, Jones T, Kuchena A, Ali S M, Hernandez-Irizarry R, Lohse C M, Farley D R. Incidence of inguinal hernia repairs in Olmsted County, M N: a population-based study. Ann Surg. 2013; 257β):520-6. doi: 10.1097/SLA.0b013e31826d41c6. PubMed PMID: 23388353; PubMed Central PMCID: PMC3761354.
23. Amato G, Agrusa A, Romano G, Salamone G, Cocorullo G, Mularo S A, Marasa S, Gulotta G. Histological findings in direct inguinal hernia: investigating the histological changes of the herniated groin looking forward to ascertain the pathogenesis of hernia disease. Hernia: the journal of hernias and abdominal wall surgery. 2013; 17(6):757-63. doi: 10.1007/s10029-012-1032-0. PubMed PMID: 23288217.
24. Amato G, Agrusa A, Romano G, Salamone G, Gulotta G, Silvestri F, Bussani R. Muscle degeneration in inguinal hernia specimens. Hernia: the journal of hernias and abdominal wall surgery. 2012; 16β):327-31. doi: 10.1007/s10029-011-0890-1. PubMed PMID: 22015811.
25. Amato G, Marasa L, Sciacchitano T, Bell S G, Romano G, Gioviale M C, Lo Monte A I, Romano M. Histological findings of the internal inguinal ring in patients having indirect inguinal hernia. Hernia: the journal of hernias and abdominal wall surgery. 2009; 13β):259-62. doi: 10.1007/s10029-009-0483-4. PubMed PMID: 19234660.
26. Malangoni M, Rosen M. Chaper 44: Hernias. In: Townsend C J,
Beauchamp D, Evers M, Mattox K, editors. Sabiston Textbook of Surgery. 18 ed. Philadelphia: Saunders Elsevier; 2008.
27. HerniaSurge G. International guidelines for groin hernia management. Hernia: the journal of hernias and abdominal wall surgery. 2018; 22(1):1-165. doi: 10.1007/s10029-017-1668-x. PubMed PMID: 29330835; PubMed Central PMCID: PMC5809582.
28. Gianetta E, de Cian F, Cuneo S, Friedman D, Vitale B, Marinari G, Baschieri G, Camerini G. Hernia repair in elderly patients. Br J Surg. 1997; 84(7):983-5. PubMed PMID: 9240142.
29. Gilbert A I. Hernia repair in the aged and infirmed. J Fla Med Assoc. 1988; 75(11):742-4. PubMed PMID: 3204359.
30. Gunnarsson U, Degerman M, Davidsson A, Heuman R. Is elective hernia repair worthwhile in old patients? Eur J Surg. 1999; 165(4):326-32. doi: 10.1080/110241599750006857. PubMed PMID: 10365833.
31. Bulun S E. Aromatase deficiency and estrogen resistance: from molecular genetics to clinic. Seminars in reproductive medicine. 2000; 18(1):31-9. PubMed PMID: 11305285.
32. Bulun S E, Lin Z, Imir G, Amin S, Demura M, Yilmaz B, Martin R, Utsunomiya H, Thung S, Gurates B, Tamura M, Langoi D, Deb S. Regulation of aromatase expression in estrogen-responsive breast and uterine disease: from bench to treatment. Pharmacological reviews. 2005; 57β):359-83. doi: 10.1124/pr.57.3.6. PubMed PMID: 16109840.
33. Bulun S E, Economos K, Miller D, Simpson E R. CYP19 (aromatase cytochrome P450) gene expression in human malignant endometrial tumors. The Journal of clinical endocrinology and metabolism. 1994; 79(6):1831-4. doi: 10.1210/jcem.79.6.7989490. PubMed PMID: 7989490.
34. Goh V H, Tong T Y, Mok H P, Said B. Interactions among age, adiposity, bodyweight, lifestyle factors and sex steroid hormones in healthy Singaporean Chinese men. Asian J Androl. 2007; 9(5):611-21. doi: 10.1111/j.1745-7262.2007.00322.x. PubMed PMID: 17712478.
35. Li J Y, Li X Y, Li M, Zhang G K, Ma F L, Liu Z M, Zhang N Y, Meng P. Decline of serum levels of free testosterone in aging healthy Chinese men. Aging Male. 2005; 8β-4):203-6. doi: 10.1080/13685530500356010. PubMed PMID: 16390747.
36. Burrows H. The occurrence of scrotal hernia in mice under treatment with oestrin. British Journal of Surgery. 1934; 21:6.
37. Gardner W U. Sexual dimorphism of the pelvis of the mouse, the effect of estrogenic hormones upon the pelvis and upon the development of scrotal hernias. American Journal of Anatomy. 1936; 59:25.
38. Hazary S, Gardner W U. Influence of sex hormones on abdominal musculature and the formation of inguinal and scrotal hernias in mice. Anat Rec. 1960; 136:437-43. PubMed PMID: 14400391.
39. Kelly M, Macdougall K, Olabisi O, McGuire N. In vivo response to polypropylene following implantation in animal models: a review of biocompatibility. Int Urogynecol J. 2017; 28(2):171-80. doi: 10.1007/s00192-016-3029-1. PubMed PMID: 27216918; PubMed Central PMCID: PMC5306078.
40. Ma X, Liu Y, Wang Q, Chen Y, Liu M, Li X, Xiang R, Wei Y, Duan Y, Han J. Tamoxifen induces the development of hernia in mice by activating MMP-2 and MMP-13 expression. Biochim Biophys Acta. 2015; 1852(5):1038-48. doi: 10.1016/j.bbadis.2015.02.006. PubMed PMID: 25703139.
41. Orenstein S B, Saberski E R, Kreutzer D L, Novitsky Y W. Comparative analysis of histopathologic effects of synthetic meshes based on material, weight, and pore size in mice. J Surg Res. 2012; 176(2):423-9. doi: 10.1016/j.jss.2011.09.031. PubMed PMID: 22099590.
42. Novitsky Y W, Orenstein S B, Kreutzer D L. Comparative analysis of histopathologic responses to implanted porcine biologic meshes. Hernia: the journal of hernias and abdominal wall surgery. 2014; 18(5):713-21. doi: 10.1007/s10029-013-1203-7. PubMed PMID: 24370604.
43. Rastegarpour A, Cheung M, Vardhan M, Ibrahim M M, Butler C E, Levinson H. Surgical mesh for ventral incisional hernia repairs: Understanding mesh design. Plast Surg (Oakv). 2016; 24(1):41-50. PubMed PMID: 27054138; PubMed Central PMCID: PMC4806756.
44. Zhao H, Innes J, Brooks D C, Reierstad S, Yilmaz M B, Lin Z, Bulun S E. A novel promoter controls Cyp19α1 gene expression in mouse adipose tissue. Reprod Biol Endocrinol. 2009; 7:37. Epub 2009/04/28. doi: 10.1186/1477-7827-7-37. PubMed PMID: 19393092; PubMed Central PMCID: PMC2684739.
45. Zhao H, Pearson E K, Brooks D C, Coon JSt, Chen D, Demura M, Zhang M, Clevenger C V, Xu X, Veenstra T D, Chatterton R T, DeMayo F J, Bulun S E. A humanized pattern of aromatase expression is associated with mammary hyperplasia in mice. Endocrinology. 2012; 153(6):2701-13. Epub 2012/04/18. doi: 10.1210/en.2011-1761. PubMed PMID: 22508516; PubMed Central PMCID: PMC3359608.
46. Wallenstein S, Zucker C L, Fleiss J L. Some statistical methods useful in circulation research. Circ Res. 1980; 47(1):1-9. Epub 1980/07/01. PubMed PMID: 7379260.
47. Bulun S E, Price T M, Aitken J, Mahendroo M S, Simpson E R. A link between breast cancer and local estrogen biosynthesis suggested by quantification of breast adipose tissue aromatase cytochrome P450 transcripts using competitive polymerase chain reaction after reverse transcription. The Journal of clinical endocrinology and metabolism. 1993; 77(6):1622-8. Epub 1993/12/01. PubMed PMID: 8117355.
48. Bulun S E, Sharda G, Rink J, Sharma S, Simpson E R. Distribution of aromatase P450 transcripts and adipose fibroblasts in the human breast. The Journal of clinical endocrinology and metabolism. 1996; 81β):1273-7. Epub 1996/03/01. PubMed PMID: 8772611.
49. Chen D, Zhao H, Coon JSt, Ono M, Pearson E K, Bulun S E. Weight gain increases human aromatase expression in mammary gland. Mol Cell Endocrinol. 2012; 355(1):114-20. Epub 2012/02/22. doi: 10.1016/j.mce.2012.01.027. PubMed PMID: 22342815; PubMed Central PMCID: PMC3312968.
50. Zhao H, Cui Y, Dupont J, Sun H, Hennighausen L, Yakar S. Overexpression of the tumor suppressor gene phosphatase and tensin homologue partially inhibits wnt-1-induced mammary tumorigenesis. Cancer research. 2005; 65(15):6864-73. Epub 2005/08/03. doi: 10.1158/0008-5472.CAN-05-0181. PubMed PMID: 16061670.
51. Bulun S E. Clinical review 78: Aromatase deficiency in women and men: would you have predicted the phenotypes? The Journal of clinical endocrinology and metabolism. 1996; 81β):867-71. doi: 10.1210/jcem.81.3.8772541. PubMed PMID: 8772541.
52. Agarwal V R, Takayama K, Van Wyk J J, Sasano H, Simpson E R, Bulun S E. Molecular basis of severe gynecomastia associated with aromatase expression in a fibrolamellar hepatocellular carcinoma. The Journal of clinical endocrinology and metabolism. 1998; 83(5):1797-800. doi: 10.1210/jcem.83.5.4773. PubMed PMID: 9589695.
53. Aida-Yasuoka K, Peoples C, Yasuoka H, Hershberger P, Thiel K, Cauley J A, Medsger T A, Jr., Feghali-Bostwick C A. Estradiol promotes the development of a fibrotic phenotype and is increased in the serum of patients with systemic sclerosis. Arthritis Res Ther. 2013; 15(1):R10. doi: 10.1186/ar4140. PubMed PMID: 23305385; PubMed Central PMCID: PMC3672719.
54. Luo N, Guan Q, Zheng L, Qu X, Dai H, Cheng Z. Estrogen-mediated activation of fibroblasts and its effects on the fibroid cell proliferation. Transl Res. 2014; 163β):232-41. doi: 10.1016/j.trs1.2013.11.008. PubMed PMID: 24316382.
55. Larionov A A, Vasyliev D A, Mason J I, Howie A F, Berstein L M, Miller W R. Aromatase in skeletal muscle. The Journal of steroid biochemistry and molecular biology. 2003; 84(4):485-92. PubMed PMID: 12732294.
56. Matsumine H, Hirato K, Yanaihara T, Tamada T, Yoshida M. Aromatization by skeletal muscle. The Journal of clinical endocrinology and metabolism. 1986; 63β):717-20. doi: 10.1210/jcem-63-3-717. PubMed PMID: 3734038.
57. Bulun S E, Simpson E R. Competitive reverse transcription-polymerase chain reaction analysis indicates that levels of aromatase cytochrome P450 transcripts in adipose tissue of buttocks, thighs, and abdomen of women increase with advancing age. The Journal of clinical endocrinology and metabolism. 1994; 78(2):428-32. doi: 10.1210/jcem.78.2.8106632. PubMed PMID: 8106632.
58. Cleland W H, Mendelson C R, Simpson E R. Effects of aging and obesity on aromatase activity of human adipose cells. The Journal of clinical endocrinology and metabolism. 1985; 60(1):174-7. doi: 10.1210/jcem-60-1-174. PubMed PMID: 3964790.
59. Hemsell D L, Grodin J M, Brenner P F, Siiteri P K, MacDonald P C. Plasma precursors of estrogen. II. Correlation of the extent of conversion of plasma androstenedione to estrone with age. The Journal of clinical endocrinology and metabolism. 1974; 38β):476-9. doi: 10.1210/jcem-38-3-476. PubMed PMID: 4815174.
60. Shozu M, Sebastian S, Takayama K, Hsu W T, Schultz R A, Neely K, Bryant M, Bulun S E. Estrogen excess associated with novel gain-of-function mutations affecting the aromatase gene. The New England journal of medicine. 2003; 348(19):1855-65. doi: 10.1056/NEJMoa021559. PubMed PMID: 12736278.
61. Harada N, Utsumi T, Takagi Y. Tissue-specific expression of the human aromatase cytochrome P-450 gene by alternative use of multiple exons 1 and promoters, and switching of tissue-specific exons 1 in carcinogenesis. Proc Natl Acad Sci USA. 1993; 90(23):11312-6. PubMed PMID: 8248245; PubMed Central PMCID: PMC47972.
62. Chen S, Zhou D, Okubo T, Kao Y C, Yang C. Breast tumor aromatase: functional role and transcriptional regulation. Endocrine-related cancer. 1999; 6(2):149-56. PubMed PMID: 10731103.
63. Fang Z, Yang S, Gurates B, Tamura M, Simpson E, Evans D, Bulun S E. Genetic or enzymatic disruption of aromatase inhibits the growth of ectopic uterine tissue. The Journal of clinical endocrinology and metabolism. 2002; 87(7):3460-6. doi: 10.1210/jcem.87.7.8683. PubMed PMID: 12107266.
64. Grant R A. Estimation of Hydroxyproline by the Autoanalyser. Journal of clinical pathology. 1964; 17:685-6. PubMed PMID: 14227441; PubMed Central PMCID: PMC480859.
65. Brown S H, Banuelos K, Ward S R, Lieber R L. Architectural and morphological assessment of rat abdominal wall muscles: comparison for use as a human model. J Anat. 2010; 217β):196-202. doi: 10.1111/j.1469-7580.2010.01271.x. PubMed PMID: 20646108; PubMed Central PMCID: PMC2919595.
66. Brown S H, Ward S R, Cook M S, Lieber R L. Architectural analysis of human abdominal wall muscles: implications for mechanical function. Spine (Phila Pa. 1976). 2011; 36(5):355-62. doi: 10.1097/BRS.0b013e3181d12ed7. PubMed PMID: 21325932; PubMed Central PMCID: PMC3017737.
67. Zhang W, Schmull S, Du M, Liu J, Lu Z, Zhu H, Xue S, Lian F. Estrogen Receptor alpha and beta in Mouse: Adipose-Derived Stem Cell Proliferation, Migration, and Brown Adipogenesis In Vitro. Cell Physiol Biochem. 2016; 38(6):2285-99. doi: 10.1159/000445583. PubMed PMID: 27197672.
68. Osborne C K, Wakeling A, Nicholson R I. Fulvestrant: an oestrogen receptor antagonist with a novel mechanism of action. Br J Cancer. 2004; 90 Suppl 1:S2-6. doi: 10.1038/sj.bjc.6601629. PubMed PMID: 15094757; PubMed Central PMCID: PMC2750773.
69. Harrington W R, Sheng S, Barnett D H, Petz L N, Katzenellenbogen J A, Katzenellenbogen B S. Activities of estrogen receptor alpha- and beta-selective ligands at diverse estrogen responsive gene sites mediating transactivation or transrepression. Mol Cell Endocrinol. 2003; 206(1-2):13-22. PubMed PMID: 12943986.
70. Al-Khyatt W, Tufarelli C, Khan R, Iftikhar S Y. Selective oestrogen receptor antagonists inhibit oesophageal cancer cell proliferation in vitro. BMC Cancer. 2018; 18(1):121. doi: 10.1186/s12885-018-4030-5. PubMed PMID: 29390981; PubMed Central PMCID: PMC5796348.
71. Fatima L A, Campello R S, Santos R S, Freitas H S, Frank A P, Machado U F, Clegg D J. Estrogen receptor 1 (ESR1) regulates VEGFA in adipose tissue. Sci Rep. 2017; 7(1):16716. doi: 10.1038/s41598-017-16686-7. PubMed PMID: 29196658; PubMed Central PMCID: PMC5711936.
72. Macedo L F, Sabnis G J, Goloubeva O G, Brodie A. Combination of anastrozole with fulvestrant in the intratumoral aromatase xenograft model. Cancer research. 2008; 68(9):3516-22. doi: 10.1158/0008-5472.CAN-07-6807. PubMed PMID: 18451180.
73. Chan K K, Leung T H, Chan D W, Wei N, Lau G T, Liu S S, Siu M K, Ngan H Y. Targeting estrogen receptor subtypes (ERalpha and ERbeta) with selective E R modulators in ovarian cancer. J Endocrinol. 2014; 221(2):325-36. doi: 10.1530/JOE-13-0500. PubMed PMID: 24819599.
74. Davis A M, Ellersieck M R, Grimm K M, Rosenfeld C S. The effects of the selective estrogen receptor modulators, methyl-piperidino-pyrazole (MPP), and raloxifene in normal and cancerous endometrial cell lines and in the murine uterus. Molecular reproduction and development. 2006; 73(8):1034-44. doi: 10.1002/mrd.20520. PubMed PMID: 16688783.
75. Han S J, Jung S Y, Wu S P, Hawkins S M, Park M J, Kyo S, Qin J, Lydon J P, Tsai S Y, Tsai M J, DeMayo F J, O'Malley B W. Estrogen Receptor beta Modulates Apoptosis Complexes and the Inflammasome to Drive the Pathogenesis of Endometriosis. Cell. 2015; 163(4):960-74. doi: 10.1016/j.cell.2015.10.034. PubMed PMID: 26544941; PubMed Central PMCID: PMC4640214.
76. Dennis M K, Burai R, Ramesh C, Petrie W K, Alcon S N, Nayak T K, Bologa C G, Leitao A, Brailoiu E, Deliu E, Dun N J, Sklar L A, Hathaway H J, Arterburn J B, Oprea T I, Prossnitz E R. In vivo effects of a GPR30 antagonist. Nat Chem Biol. 2009; 5(6):421-7. doi: 10.1038/nchembio.168. PubMed PMID: 19430488; PubMed Central PMCID: PMC2864230.
77. Mohammed H, Russell I A, Stark R, Rueda O M, Hickey T E, Tarulli G A, Serandour A A, Birrell S N, Bruna A, Saadi A, Menon S, Hadfield J, Pugh M, Raj G V, Brown G D, D'Santos C, Robinson J L, Silva G, Launchbury R, Perou C M, Stingl J, Caldas C, Tilley W D, Carroll J S. Progesterone receptor modulates ERalpha action in breast cancer. Nature. 2015; 523(7560):313-7. doi: 10.1038/nature14583. PubMed PMID: 26153859; PubMed Central PMCID: PMC4650274.
78. Barski A, Cuddapah S, Cui K, Roh T Y, Schones D E, Wang Z, Wei G, Chepelev I, Zhao K. High-resolution profiling of histone methylations in the human genome. Cell. 2007; 129(4):823-37. doi: 10.1016/j.cell.2007.05.009. PubMed PMID: 17512414.
79. Cedar H, Bergman Y. Linking DNA methylation and histone modification: patterns and paradigms. Nat Rev Genet. 2009; 10(5):295-304. doi: 10.1038/nrg2540. PubMed PMID: 19308066.
80. Koch C M, Andrews R M, Flicek P, Dillon S C, Karaoz U, Clelland G K, Wilcox S, Beare D M, Fowler J C, Couttet P, James K D, Lefebvre G C, Bruce A W, Dovey O M, Ellis P D, Dhami P, Langford C F, Weng Z, Birney E, Carter N P, Vetrie D, Dunham I. The landscape of histone modifications across 1% of the human genome in five human cell lines. Genome research. 2007; 17(6):691-707. doi: 10.1101/gr.5704207. PubMed PMID: 17567990; PubMed Central PMCID: PMC1891331.
81. Liu X, Wang C, Liu W, Li J, Li C, Kou X, Chen J, Zhao Y, Gao H, Wang H, Zhang Y, Gao Y, Gao S. Distinct features of H3K4me3 and H3K27me3 chromatin domains in pre-implantation embryos. Nature. 2016; 537(7621):558-62. doi: 10.1038/nature19362. PubMed PMID: 27626379.
82. Creyghton M P, Cheng A W, Welstead G G, Kooistra T, Carey B W, Steine E J, Hanna J, Lodato M A, Frampton G M, Sharp P A, Boyer L A, Young R A, Jaenisch R. Histone H3K27ac separates active from poised enhancers and predicts developmental state. Proc Natl Acad Sci USA. 2010; 107(50):21931-6. doi: 10.1073/pnas.1016071107. PubMed PMID: 21106759; PubMed Central PMCID: PMC3003124.
83. Kalluri R. The biology and function of fibroblasts in cancer. Nat Rev Cancer. 2016; 16(9):582-98. doi: 10.1038/nrc.2016.73. PubMed PMID: 27550820.
84. Hewitt S C, Li L, Grimm S A, Chen Y, Liu L, Li Y, Bushel P R, Fargo D, Korach K S. Research resource: whole-genome estrogen receptor alpha binding in mouse uterine tissue revealed by ChIP-seq. Mol Endocrinol. 2012; 26(5):887-98. doi: 10.1210/me.2011-1311. PubMed PMID: 22446102; PubMed Central PMCID: PMC3355558.
85. Lin Z, Reierstad S, Huang C C, Bulun S E. Novel estrogen receptoralpha binding sites and estradiol target genes identified by chromatin immunoprecipitation cloning in breast cancer. Cancer research. 2007; 67(10):5017-24. Epub 2007/05/19. doi: 10.1158/0008-5472. CAN-06-3696. PubMed PMID: 17510434.
86. Dyson M T, Roqueiro D, Monsivais D, Ercan C M, Pavone M E, Brooks D C, Kakinuma T, Ono M, Jafari N, Dai Y, Bulun S E. Genome-wide DNA methylation analysis predicts an epigenetic switch for GATA factor expression in endometriosis. PLoS genetics. 2014; 10β):e1004158. doi: 10.1371/journal.pgen.1004158. PubMed PMID: 24603652; PubMed Central PMCID: PMC3945170.
87. Yin P, Roqueiro D, Huang L, Owen J K, Xie A, Navarro A, Monsivais D, Coon JSt, Kim J J, Dai Y, Bulun S E. Genome-wide progesterone receptor binding: cell type-specific and shared mechanisms in T47D breast cancer cells and primary leiomyoma cells. PloS one. 2012; 7(1):e29021. Epub 2012/01/25. doi: 10.1371/journal.pone.0029021. PubMed PMID: 22272226; PubMed Central PMCID: PMC3260146.
88. Zhang Y, Liu T, Meyer C A, Eeckhoute J, Johnson D S, Bernstein B E, Nusbaum C, Myers R M, Brown M, Li W, Liu X S. Model-based Analysis of ChIP-Seq (MACS). Genome Biology. 2008; 9(9):R137. doi: 10.1186/gb-2008-9-9-r137.
89. Lun A T L, Smyth G K. csaw: a Bioconductor package for differential binding analysis of ChIP-seq data using sliding windows. Nucleic Acids Research. 2015; 44(5):e45-e. doi: 10.1093/nar/gkv1191.
90. Bray N, Pimentel H, Melsted P, Pachter L. Near-optimal probabilistic RNA-Seq quantification. Nat Biotechnol. 2016.
91. Robinson M D, McCarthy D J, Smyth G K. edgeR: a Bioconductor package for differential expression analysis of digital gene expression data. Bioinformatics. 2010; 26. doi: 10.1093/bioinformatics/btp616.
92. Wang S, Sun H, Ma J, Zang C, Wang C, Wang J. Target analysis by integration of transcriptome and ChIP-seq data with BETA. Nat Protoc. 2013; 8. doi: 10.1038/nprot.2013.150.
93. Wang S, Sun H, Ma J, Zang C, Wang C, Wang J, Tang Q, Meyer C A, Zhang Y, Liu X S. Target analysis by integration of transcriptome and ChIP-seq data with BETA. Nat Protoc. 2013; 8(12):2502-15. Epub 2013/11/23. doi: 10.1038/nprot.2013.150 nprot.2013.150 [pii]. PubMed PMID: 24263090; PubMed Central PMCID: PMC4135175.
94. Welboren W J, van Driel M A, Janssen-Megens E M, van Heeringen S J, Sweep F C, Span P N, Stunnenberg H G. ChIP-Seq of ERalpha and RNA polymerase II defines genes differentially responding to ligands. EMBO J. 2009; 28(10):1418-28. doi: 10.1038/emboj.2009.88. PubMed PMID: 19339991; PubMed Central PMCID: PMC2688537.
95. Lin Z, Yin P, Reierstad S, O'Halloran M, Coon V J, Pearson E K, Mutlu G M, Bulun S E. Adenosine A1 receptor, a target and regulator of estrogen receptoralpha action, mediates the proliferative effects of estradiol in breast cancer. Oncogene. 2010; 29(8):1114-22. Epub 2009/11/26. doi: 10.1038/onc.2009.409. PubMed PMID: 19935720; PubMed Central PMCID: PMC2829108.
96. Yin P, Lin Z, Cheng Y H, Marsh E E, Utsunomiya H, Ishikawa H, Xue Q, Reierstad S, Innes J, Thung S, Kim J J, Xu E, Bulun S E. Progesterone receptor regulates Bcl-2 gene expression through direct binding to its promoter region in uterine leiomyoma cells. The Journal of clinical endocrinology and metabolism. 2007; 92(11):4459-66. Epub 2007/09/06. doi: 10.1210/jc.2007-0725. PubMed PMID: 17785366.
97. Yin P, Lin Z, Reierstad S, Wu J, Ishikawa H, Marsh E E, Innes J, Cheng Y, Pearson K, Coon JSt, Kim J J, Chakravarti D, Bulun S E. Transcription factor KLF11 integrates progesterone receptor signaling and proliferation in uterine leiomyoma cells. Cancer research. 2010; 70(4):1722-30. Epub 2010/02/04. doi: 10.1158/0008-5472.CAN-09-2612. PubMed PMID: 20124487; PubMed Central PMCID: PMC2822899.
98. Ono M, Yin P, Navarro A, Moravek M B, Coon JSt, Druschitz S A, Sema V A, Qiang W, Brooks D C, Malpani S S, Ma J, Ercan C M, Mittal N, Monsivais D, Dyson M T, Yemelyanov A, Maruyama T, Chakravarti D, Kim J J, Kurita T, Gottardi C J, Bulun S E. Paracrine activation of WNT/beta-catenin pathway in uterine leiomyoma stem cells promotes tumor growth. Proc Natl Acad Sci USA. 2013; 110(42):17053-8. doi: 10.1073/pnas.1313650110. PubMed PMID: 24082114; PubMed Central PMCID: PMC3801037.
99. Yin P, Roqueiro D, Huang L, Owen J K, Xie A, Navarro A, Monsivais D, Coon V J S, Kim J J, Dai Y, Bulun S E. Genome-Wide Progesterone Receptor Binding: Cell Type-Specific and Shared Mechanisms in T47D Breast Cancer Cells and Primary Leiomyoma Cells. PLoS ONE. 2012; 7(1):e29021. doi: 10.1371/journal.pone.0029021.
100. Xu J, Hu H, Dai Y. LMethyR-SVM: Predict Human Enhancers Using Low Methylated Regions based on Weighted Support Vector Machines. PLoS ONE. 2016; 11(9):e0163491. doi: 10.1371/journal.pone.0163491.
101. Larsen P E, Sreedasyam A, Trivedi G, Shinde S V, Dai Y, Cseke L J, Collart F R. Multi-omics approach identifies molecular mechanisms of plant-fungus mycorrhizal interaction. Frontiers in Plant Science. 2016; 6.
102. Hong H, Jingling X, Yang D. Regulatory Elements in Low-Methylated Regions Predict Directional Change of Gene Expression. Biomedical and Health Informatics, IEEE Journal of 2015; 19(4):1293-300. doi: 10.1109/JBHI.2015.2431640.
103. Feder D, Rodrigues Barros Godoy I, Guimaraes Pereira M L, Silva C S, Nogueira Silvestre D, Fonseca F L, Alves de Siqueira Carvalho A, Aparecida dos Santos R, Catteli Carvalho M H. Hormonal receptors in skeletal muscles of dystrophic mdx mice. Biomed Res Int. 2013; 2013:604635. doi: 10.1155/2013/604635. PubMed PMID: 23509749; PubMed Central PMCID: PMC3591173.
104. Wiik A, Ekman M, Johansson O, Jansson E, Esbjornsson M. Expression of both oestrogen receptor alpha and beta in human skeletal muscle tissue. Histochem Cell Biol. 2009; 131(2):181-9. doi: 10.1007/s00418-008-0512-x. PubMed PMID: 18825402.
105. Luther E R, Roux J, Popat R, Gardner A, Gray J, Soubiran E, Korcaz Y. The effect of estrogen priming on induction of labor with prostaglandins. Am J Obstet Gynecol. 1980; 137β):351-7. PubMed PMID: 7377255.
106. Iwano M, Plieth D, Danoff T M, Xue C, Okada H, Neilson E G. Evidence that fibroblasts derive from epithelium during tissue fibrosis. The Journal of clinical investigation. 2002; 110β):341-50. doi: 10.1172/JCI15518. PubMed PMID: 12163453; PubMed Central PMCID: PMC151091.
107. Chen M, Wolfe A, Wang X, Chang C, Yeh S, Radovick S. Generation and characterization of a complete null estrogen receptor alpha mouse using Cre/LoxP technology. Molecular and cellular biochemistry. 2009; 321(1-2):145-53. doi: 10.1007/s11010-008-9928-9. PubMed PMID: 18953638; PubMed Central PMCID: PMC4696932.
108. Chen D, Reierstad S, Fang F, Bulun S E. JunD and JunB integrate prostaglandin E2 activation of breast cancer-associated proximal aromatase promoters. Mol Endocrinol. 2011; 25(5):767-75. doi: 10.1210/me.2010-0368. PubMed PMID: 21393445; PubMed Central PMCID: PMC3082330.
109. Chen D, Reierstad S, Lin Z, Lu M, Brooks C, Li N, Innes J, Bulun S E. Prostaglandin E(2) induces breast cancer related aromatase promoters via activation of p38 and c-Jun NH(2)-terminal kinase in adipose fibroblasts. Cancer research. 2007; 67(18):8914-22. Epub 2007/09/19. doi: 10.1158/0008-5472.CAN-06-4751. PubMed PMID: 17875734.
110. Zhou J, Gurates B, Yang S, Sebastian S, Bulun S E. Malignant breast epithelial cells stimulate aromatase expression via promoter II in human adipose fibroblasts: an epithelial-stromal interaction in breast tumors mediated by CCAAT/enhancer binding protein beta. Cancer research. 2001; 61(5):2328-34. PubMed PMID: 11280806.
111. Deb S, Jianfeng Z, Amin S A, Gonca I A, Bertan Y M, Zihong L, Bulun S E. A novel role of sodium butyrate in the regulation of cancer-associated aromatase promoters I.3 and II by disrupting a transcriptional complex in breast adipose fibroblasts. J Biol Chem. 2006; 281(5):2585-97. PubMed PMID: 16303757.
112. Li X, et al., “Altered Structure and Function of Reproductive Organs in Transgenic Male Mice Overexpressing Human Aromatase*” Endocrinology (2001), Vol. 142, No. 6, pages 2435-2442.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for treating and/or preventing a hernia in a subject in need thereof, the method comprising administering to the subject one or more therapeutic agents that modulate the production and/or activity of one or more sex steroid hormones in the subject.

2. The method of claim 1, wherein the subject has or is at risk for developing a hernia selected from an inguinal hernia, a femoral hernia, an umbilical hernia, a hiatal hernia, an incisional hernia, and diastasis recti.

3. The method of claim 1, wherein the subject has or is at risk for developing an inguinal hernia.

4. The method of claim 1, wherein the therapeutic agent inhibits the production and/or activity of estrogen.

5. The method of claim 4, wherein the therapeutic agent is an anti-estrogen therapeutic that inhibits the activity of estrogen in the subject.

6. The method of claim 5, wherein the anti-estrogen therapeutic is a selective estrogen receptor modulator or degrader (SERM or SERD).

7. The method of claim 6, wherein the SERM or SERD is raloxifene or fulvestrant.

8. The method of claim 4, wherein the therapeutic agent inhibits the production of estrogen in the subject.

9. The method of claim 8, wherein the therapeutic agent is an aromatase inhibitor.

10. The method of claim 9, wherein the aromatase inhibitor is letrozole.

11. The method of claim 1, wherein the therapeutic agent stimulates the production and/or activity of androgens in the subject.

12. The method of claim 11, wherein the therapeutic agent is an androgen or an androgen stimulator.

13. The method of claim 12, wherein the androgen or androgen stimulator is testosterone, dihydrotestosterone, 19-Nortestosterone, 17α-alkylated testostereon, 17α-alkylated dihydrotestosteron, 17α-alkylated 19-nortestosterone, 17α-vinylated testosterone, 17α-ethynylated testosterone, R1881, human chorionic gonadotropin, or an analogue or derivative thereof.

14. The method of claim 1, wherein the therapeutic agent is administered locally to the subject at the hernia or at a site at risk for developing a hernia.

15. The method of claim 1, wherein the therapeutic agent is administered topically.

16. The method of claim 1, wherein the subject has undergone surgery for hernia repair or the subject is preparing to undergo surgery for hernia repair and the therapeutic agent is administered to the subject before, during, and/or after surgery.

17. The method of claim 1, wherein the subject has undergone surgery and the therapeutic agent is incorporated into a hernia repair mesh material that is implanted in the subject at the hernia and that releases the therapeutic agent to the hernia.

18. The method of claim 1, wherein the therapeutic agent is administered systemically.

19. The method of claim 1, wherein the therapeutic agent is administered orally.

20. The method of claim 1, wherein the therapeutic agent is administered via injection.
